(19) United States
(12) Patent Application Publication
(10) Pub. No.: US 2006/0235201 A1
(43) Pub. Date: Oct. 19, 2006

(54) Enduring T cell response

(76) Inventor: Roman Kischel, Zorneding (DE)

Correspondence Address: FULBRIGHT & JAWORSKI L.L.P., 600 CONGRESS AVE., SUITE 2400, AUSTIN, TX, 78701, US

(21) Appl. No.: 10/544,589

(22) PCT Filed: Feb. 5, 2004
(86) PCT No.: PCT/EP04/01068

§ 371 Date: Mar. 9, 2006

(30) Foreign Application Priority Data
Feb. 6, 2003 (EP) 03002741.1

(51) Int. Cl. C07K 14/82; C12Q 1/68; C07H 21/04; C12P 21/06; C07K 16/30; A61K 39/00; A61K 48/00

(52) U.S. Cl. 530/350; 530/388.8; 435/6; 435/69.1; 435/320.1; 435/325; 514/12; 536/23.5; 514/44

(57) ABSTRACT

The present invention provides a trimeric polypeptide construct, wherein each monomer of the trimeric polypeptide construct consists of two or three domains, and wherein the first domain is the extracellular domain of 4-1BBL or (a) part(s) thereof, the second domain consists of an antigen-interaction-site which is located N-terminally of the first domain and, optionally, the third domain combines said first and second domain via a peptide linker, wherein said peptide linker does not comprise any polymerization activity. Further, the invention provides nucleic acid molecules encoding said polypeptide constructs, vectors and hast systems for the expression of the trimeric polypeptide construct. Moreover, the invention provides compositions which are envisaged to be pharmaceutical compositions and their use in the treatment of diseases.

The present invention relates to a trimeric polypeptide construct, wherein each monomer of the trimeric polypeptide construct consists of two or three domains, and wherein the first domain is the extracellular domain of 4-1BBL or (a) part(s) thereof, the second domain consists of an antigen-interaction-site which is located N-terminally of the first domain and, optionally, the third domain combines said first and second domain via a peptide linker, wherein said peptide linker does not comprise any polymerization activity. Further, the invention provides nucleic acid molecules encoding said polypeptide constructs, vectors and host systems for the expression of the trimeric polypeptide construct. Moreover, the invention provides compositions which are envisaged to be pharmaceutical compositions and their use in the treatment of diseases. A variety of documents is cited throughout this specification. The disclosure content of said documents is herewith incorporated by reference.

The improvement of T cell mediated immunotherapy is an important medical goal. One key aspect in T cell mediated immunotherapy is efficient T cell activation. T cell activation results from the dynamic interaction of antigen by contact with antigen displayed on the membrane of an antigen presenting cell (APC) and multiple membrane molecules generating the necessary intracellular signals. It is now widely believed that naive T cells require several distinct signals for activation and subsequent proliferation into effector cells.

First, there is an initial signal (signal 1), which is generated by the interaction of an antigenic peptide with the TCR-CD3 complex (Kuby, 2000: Immunology, 4th edition by Freeman and Company, N.Y., page 254). This initial signal leads to a short term stimulation of the T cells. The complementary determining region (CDR) dictates the antigen specificity of the response and plays a central role in initiating activation. However, this interaction, by itself, is not sufficient to fully activate naive T cells. After the initial T cell stimulation there have to be further, antigen-independent costimulatory signals.

The second signal is mediated by the interaction between B7.1 and the CD28 receptor on T cells. The CD28 receptor is expressed already on resting/naive T cells and it mediates T cell proliferation and the actual priming of the T cells. The third signal is generated by the interaction between a accessory factor like 4-1BBL and its corresponding receptor, which is expressed on the T cells only after initial stimulation. Other accessory factor/receptor pairs are: CD30 ligand/CD30, Ox40 ligand/Ox40, GITRL/GITR, CD27 ligand/CD27. This accessory signal determines the survival and fate of the T cells. The above-mentioned molecules are part of TNF superfamily.

Most TNF family members are synthesized as precursor molecules, which undergo a processing step (Bodmer et al, 2002, TIBS 27, 19-26). Processing of tumor necrosis factor alpha precursor by metalloproteinases has been described in detail (Gearing et al., 1994, Nature 370, 555-557). TNFα is initially expressed as 233 amino acid membrane anchored precursor, which is proteolytically processed to yield the mature 157aa cytokine. The processing enzyme, which cleaves TNFα is tumor necrosis factor a coverting enzyme TACE (Lee, Biochem. J. 2003 Jan 30, Becker et al. 2002, Biol. Chem. 383:1921-6). TACE is a membrane-anchored, multiple-domain zinc metalloproteinase responsible for the release of the potent pro-inflammatory cytokine, TNF-alpha (Lee, Biochem. J. 2003 Jan. 30). TNFα itself has been described to be a trimeric protein (Jones et al., 1990, J. Cell Sci Suppl 13, 11-18, Smith & Baglioni, 1987, J Biol Chem 262, 6951-6954; Wingfield et al., 1987, FEBS 211(2), 179-184). This is an important biological feature, since one TNFα trimere can bind three TNFα receptor molecules, and this receptor clustering initiates the intracellular signalling cascade (Ameloot et al. J. Biol. Chem (2001) 276:27098-27103). Besides the trimerization of TNFα itself, the trimerization of TNFα fusion proteins has been described. However, the trimere formation in the prior art studies was absent or low and an additional trimerization domain was used. For example, Yang et al., 1995, Mol. Immunol 32, 873-81, Scherf et al., 1996, Clin Cancer Res 9, 1523-31, Wüest et al., 2002, Oncogene 21, 4257-4265 and Xiang et al., 1997, J Biotech 53, 3-12 describe trimerization of constructs comprising the mature extracellular domain of TNFα (amino acids 1-157) and Wüest et al., 2002, Oncogene 21, 4257-4265, WO 02/22833 and WO 02/22680 describe trimerization of polypeptide constructs comprising TNFα or a TNFα homologue TRAIL or FasL with the help of an additional trimerization domain (tenascin or Acrp30).

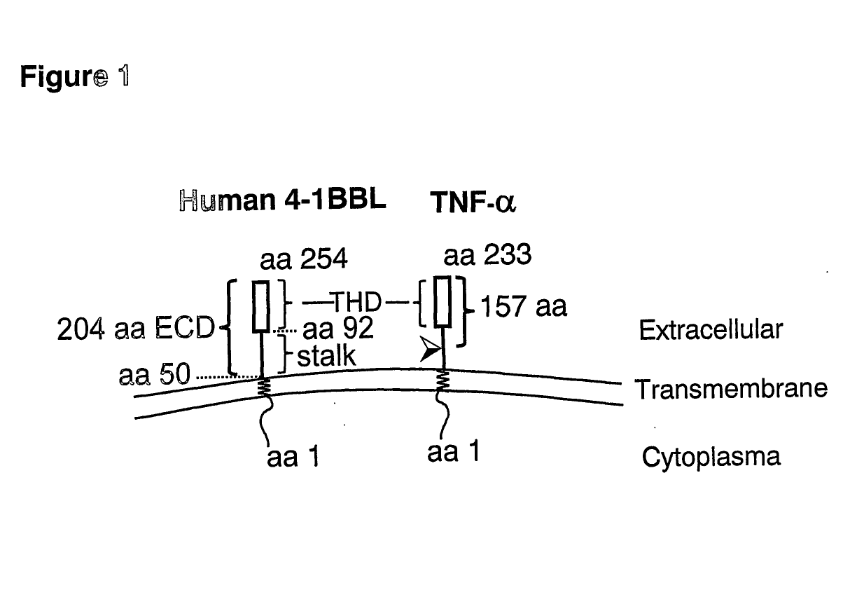

Human 4-1BBL (4-1BBL) is a type II membrane glycoprotein with an extracellular carboxyterminal domain (Goodwin et al., 1993, Eur J Immunol 23, 2631-2641). The interaction of 4-1BBL with its corresponding receptor 4.1BB induces the proliferation of activated thymocytes and splenic T cells (Goodwin et al., 1993, Eur J Immunol 23, 2631-2641). The extracellular domain of 4-1BBL shows clear sequence homology to the extracellular region of tumor necrosis factor (TNF) a. This so-called TNF homology domain (THD) is conserved between a total number of 18 TNF family members, which are known so far (Bodmer et al, 2002, TIBS 27, 19-26). The THD is an about 150 amino acid long sequence containing a conserved framework of aromatic and hydrophobic residues (Bodmer et al, 2002, TIBS 27, 19-26; Gruss, 1996, Int J Clin Lab Res 26, 143-159). THDs share a virtually identical tertiary fold and associate to form trimeric proteins (Jones et al., 1990, J. Cell Sci Suppl 13, 11-18; Smith & Baglioni, 1987, J Biol Chem 262, 6951-6954; Wingfield et al., 1987, FEBS 211(2),179-184). The THDs are beta-sandwich structures, which adopt a classical “jelly-roll” topology. From X-ray crystallography it becomes clear that the subunits associate tightly about a threefold axis interacting through a simple edge-to-face packing of the beta-sandwich to form the solid, conical shaped trimer (Jones et al., 1990, J. Cell Sci Suppl 13, 11-18). The amino acid residues, which are responsible for the receptor binding are buried within the THD domain (Bodmer et al, 2002, TIBS 27, 19-26). Besides the THD domain, the extracellular part of human 4-1BBL (amino acid 50-254) contains an additional stalk domain of about 42 amino acids (amino acids 50-92). FIG. 1 shows a schematic figure of the whole structure of 4-1BBL in comparison to TNF alpha and the TNF alpha precursor protein. No cleavage site has been described for 4-1 BB ligand (Bodmer et al., 2002 TIBS 27(1),19-26)

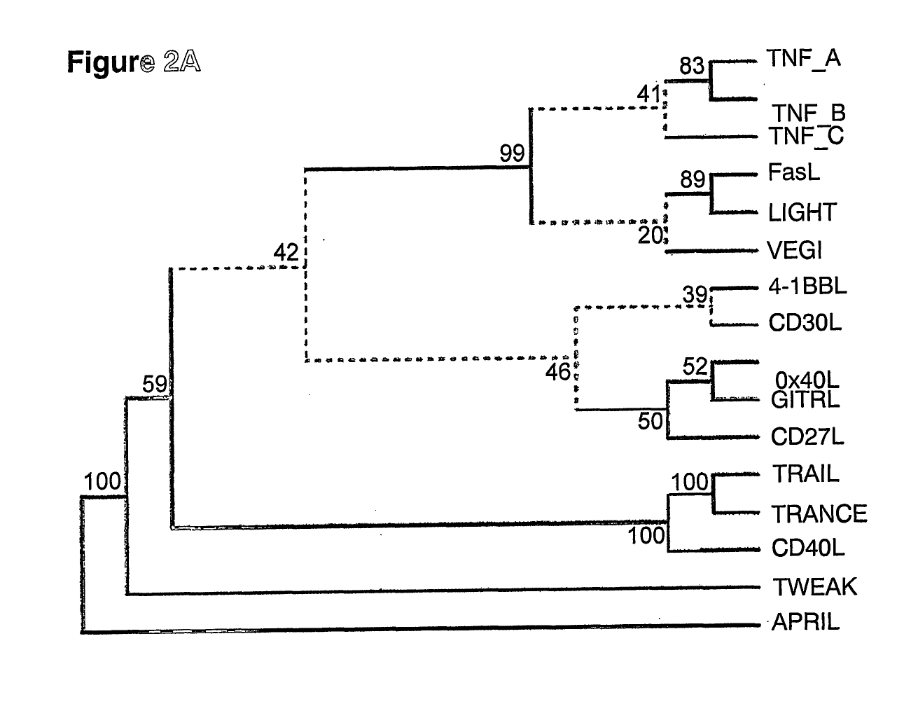

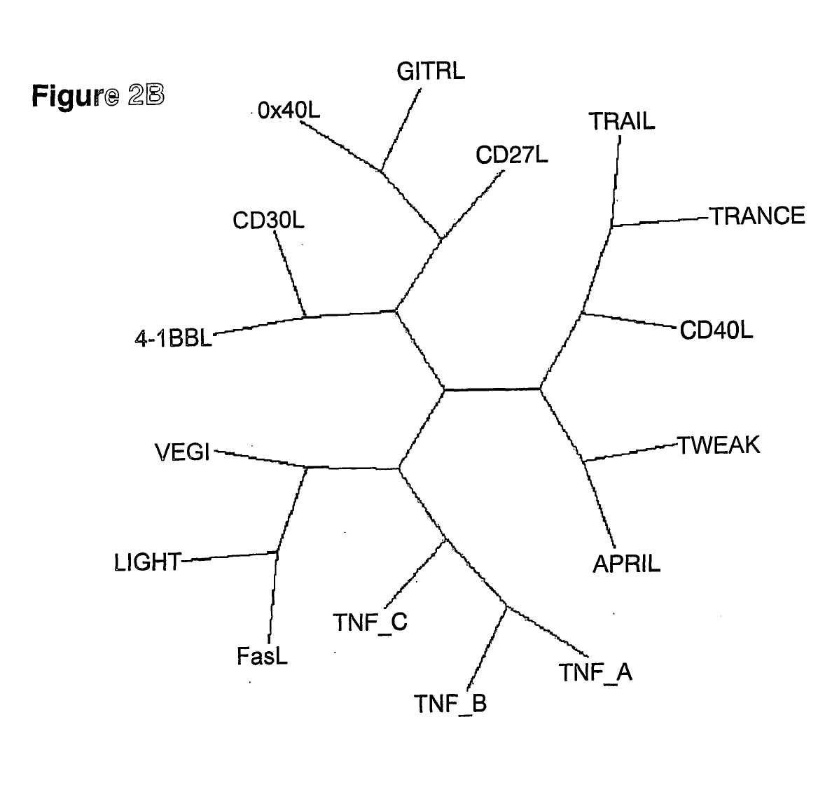

4.1BB ligand, CD30 ligand, Ox40 ligand, GITRL, LIGHT and CD27 ligand are described as having T cell (co)stimulatory or regulating function (Mackay & Kalled, 2002, Current Opinion in Immunology 14, 783, Granger, 2001, J. Immunol., 5122; Akiba, 2000, J. Exp. Med. 191, 375) and therefore, form a subgroup in the TNF ligand superfamily distinct from ligands which act on B cells or dendritic cells. These reports on a subpopulation of TNF family members are further supported by multiple sequence alignments and phylogenetic tree analysis. 4-1BBL lies on the same branch of the phylogenetic tree as Ox40 ligand and CD27 ligand do, whereas TNF and FasL lie on a separate branch (Granger, 2001, J. Imminol., 5122; Akiba, 2000, J. Exp. Med. 191, 375). In FIG. 2 the extracellular domains of all 18 TNF alpha family members have been compared (FIG. 2). The topological algorithm of the Treetop program calculates the pairwise distances between the sequences (Chumakov & Yushmanov, 1988, Mol Genet Microbiol Virusol 3, 3-9; Yushmanov & Chumakov, 1988, Mol Genet Microbiol Virusol 3, 9-15; Brodsky et al., 1992, Dimacs 8, 127-139; Brodsky et al., 1995, Biochemistry, 923-928). The unrooted tree appears divided into three major branches (FIG. 2B). In one of the three branches 4-1BBL groups together with CD30 ligand, Ox40 ligand, GITRL, und CD27 ligand.

It is only after a T cell has received all three signals that a lasting immune response of these T cells is generated.

T cell mediated immunotherapy, so far, has focused mainly on providing for the initial T cell stimulus. An example are the bispecific single chain antibody constructs, which generate an initial T cell stimulus via their anti-CD3 portion (Mack et al., 1995, PNAS 92(15), 7021-5; WO99/54440) or the OKT3 antibody (U.S. Pat. No. 5,929,212, WO91/09968). Components, which act via anti-CD3 loose their T cell stimulatory capacity soon after the administration. This characteristic may be used for example in an acute therapy setting. However, there are also indications, where an enduring T cell response may be desirable such as in metastatic cancer or in the treatment of minimal residual cancer.

To develop immunotherapies, which provoke an enduring T cell response, several approaches are known. However, none of them can be used to treat, ameliorate or prevent a specific condition in a target tissue.

For example, WO 99/36093 discloses a method of enhancing T cell activation comprising administration of an effective amount of a human 4-1BB ligand such that said ligand comes into contact with at least one T cell, thereby activating it. Additionally, it is defined that in the method a second stimulatory molecule may be administered in conjunction with the 4-1BB ligand. This second stimulatory molecule may be a CD3 antibody, a CD28 antibody or the CD28 protein. Optionally, if the second stimulatory molecule is a CD3 antibody, the method of WO 99/36093 may comprise a third stimulatory molecule, which may be a CD28 antibody. In particular, WO 99/36093 describes that the coengagement of CD28 with 4-1 BB promotes type 1 effector T cell development and long-term cell survival for cells susceptible to apoptosis induced by repeated TCR activation.

In WO94/26290 the DNA and the encoded amino acid sequences of 4-1BB ligand, a fusion protein comprising the 4-1BB ligand and an Fc domain has been described. In W094/26290 it is discussed that 4-1BB ligand may be used to stimulate proliferation of activated T-cells that are to be employed in therapeutic procedures and to enhance proliferation of the CTLs in the ex vivo stage, by adding 4-1BB-L to the culture medium, either alone or in combination with other cytokines such as interleukin-2.

WO98/16249 describes two anti-4-1BB monoclonal antibodies, which provide a novel approach to immunosuppression and cancer therapy in vivo.

Thus, the technical problem underlying the present invention was to provide means for an enduring/long lasting activation of T cells which may be used in the therapy of several diseases.

The solution to said technical problem is achieved by providing the embodiments characterized in the claims.

Accordingly, the present invention relates to a trimeric polypeptide construct, wherein each monomer of the trimeric polypeptide construct consists of two or three domains, and wherein the first domain is the extracellular domain of 4-1BBL or (a) part(s) thereof, the second domain consists of an antigen-interaction-site which is located N-terminally of the first domain and, optionally, the third domain combines said first and second domain via a peptide linker, wherein said peptide linker does not comprise any polymerization activity.

The term “polypeptide construct(s)” defines, in accordance with the present invention, (a) recombinant producible polypeptide(s) which are encoded by one or more genetically engineered nucleic acid molecules.

The term “trimeric polypeptide construct” as used herein denotes a construct comprising three “monomeric” polypeptide constructs. Each of said monomers of the trimeric polypeptide construct consists of at least one polypeptide chain. Thus, the term “monomeric polypeptide constructs” merely designates herein the subunits which form the “trimeric polypeptide construct” although said monomers itself may be polymers. An example for a polymer which is defined as a monomer of the trimeric construct is a F(ab) fragment which consists of two polypeptide chains. Preferably, the trimeric polypeptide constructs of the invention are soluble polypeptide constructs which may be expressed in the cytosol of an appropriate host. Likewise preferably, said polypeptide constructs of the invention are secreted into specific cellular compartments or into the supernatant via a secretory pathway of an appropriate host. A particular preferred host is an eukaryotic host. The trimeric structure of the polypeptide construct of the invention represents an essential technical feature, since it has been surprisingly found that only said trimeric structure enables the induction of activation signals which allows a persistent and/or enduring T-cell response.

The term “enduring T cell response” means that T cells have been primed through a TCR- or TCR-like signal and a second and/or third costimulatory signal. Said T cells involved in an enduring T cell response in accordance with this invention show prolonged survival. Without being bound by theory, said prolonged survival may be due to protection against activation-induced cell death and the like. As a result, activated T cells involved in enduring T cell response are, in context of this invention, available for prolonged periods of time to act as effector cells on their respective targets. The effect on T cell survival may be analysed by measuring the increase of the expression level of antiapoptotic factors e.g. from the Bcl-2 family like Bclw, Bcl-2, Bcl-x_{L} or Bfl-1 (Jones (2000) J. Exp. Med. 191: 1721).

Consequently, the trimerization of the monomers which form the trimeric polypeptide constructs of the invention represents a necessity for the function of the inventive constructs.

The term “domain” as used herein describes a subunit of a monomer of the trimeric polypeptide construct. Said domains represent regions of the polypeptide which are defined by specific technical features, e.g. the capacity to bind specific to an antigen, to promote the formation of the trimeric structure or to link separate domains with each other.

As described herein above, 4-1BBL is a type II transmembrane protein which is a member of the TNF superfamily. Complete or full length 4-1BBL has been described to form homotrimers on the surface of cells. The formation of the homotrimers is enabled by specific motives of the extracellular domain of 4-1BBL. Said motives are designated herein as “trimerization region”.

The term “extracellular domain of 4-1BBL” relates to specific motives of the extracellular domain of 4-1BBL which enable the described surprising formation of homotrimers of 4-1BBL. Accordingly, said term relates to (a) trimerization region(s) of the extracellular domain of 4-1BBL, i.e. also to part(s) or fragment(s) of said extracellular domain. The person skilled in the art is easily in the position, consulting the teaching of the appended examples, to determine functional part(s) or fragment(s) of the extracellular domain of 4-1BBL. Functional part(s) or fragment(s) are defined as being capable of trimerization.

As described above, 4-1BBL is one member in a family of proteins of which TNF is the naming and leading member. Xiang et al. (J. Biotech. (1997) 53, 3-9) have described a construct of a TNF-fusion protein which is secreted by transfected mammalian cells only in the format of a dimer; see FIG. 2 in Xiang et al. Thus, the capacity of the trimerization regions of the extracellular domain of 4-1BBL to be sufficient to trimerize the constructs of the invention is especially surprising for constructs expressed in eukaryotic cells.

As documented in the appended examples, it was surprisingly found that the capacity of the trimerization region of the extracellular domain of 4-1BBL alone is sufficient for the quantitative trimerization of complex fusion proteins (no monomers or dimers detectable). This sufficiency was neither disclosed nor expected by the prior art. In contrast, the prior art has speculated that additional trimerization domains are required. Such additional trimerization domains in complex TNFα fusion constructs have been described to be tenascin or other peptide linkers, which have trimerization capacity (WO 02/22833). In the constructs of the present invention, however, there is no need for such an additional peptide linker to induce quantitative trimer formation of the complex 4-1BBL fusion proteins described herein. Therefore, the constructs of the present invention consist of three monomers, each monomer consisting of two or three domains, whereby one of said domains is the extracellular domain of 4-1BBL. The second or third domain is not and does not comprise any trimerization domain or a polypeptide linker with polymerization activity.

The term “antigen-interaction-site” defines, in accordance with the present invention, a motive of a polypeptide which shows the capacity of specific interaction with a specific antigen or a specific group of antigens. The “interaction” of said “antigen-interaction-site” with an antigen is specific and characterized by a high binding constant of ≦10⁻⁹ M. In contrast, an unspecific interaction with an antigen is characterized by an extremely low binding constant of ≧10⁻⁵ M. The specific interaction of the antigen-interaction-site with its specific antigen may result in an initiation of a signal, e.g. due to the induction of a change of the conformation of the antigen, an oligomerization of the antigen, etc. Said binding may be exemplified by the specificity of a “key-lock-principle”. Thus, specific motives in the amino acid sequence of the antigen-interaction-site and the antigen bind to each other as a result of their primary, secondary or tertiary structure as well as the result of secondary modifications of said structure. The specific interaction of the antigen-interaction-site with its specific antigen may result as well in a simple binding of said site to the antigen.

Examples for the specific interaction of an antigen-interaction-site with a specific antigen comprise the specificity of a ligand for its receptor. Said definition particularly comprises the interaction of ligands which induce a signal upon binding to its specific receptor. Examples for corresponding ligands comprise cytokines which interact/bind with/to its specific cytokine-receptors. Also particularly comprised by said definition is the binding of an antigen-interaction-site to antigens like antigens of the selectin family, integrins and of the family of growth factors like EGF. An other example for said interaction, which is also particularly comprised by said definition, is the interaction of an antigenic determinant (epitope) with the antigenic binding site of an antibody.

The second domain is located N-terminally of the first domain. Consequently, in a nucleic acid molecule encoding a monomer of the trimeric polypeptide of the invention the coding region for the second domain is 5! of the coding sequence for the first domain.

The term “peptide linker” defines in accordance with the present invention an amino acid sequence by which the amino acid sequences of the first domain and the second domain of the monomer of the trimeric polypeptide construct of the invention are linked with each other. An essential technical feature of such peptide linker is that said peptide linker does not comprise any polymerization activity. A particularly preferred peptide linker is characterized by the amino acid sequence Gly—Gly—Gly—Gly—Ser, i.e. (Gly)₄Ser, or polymers thereof, i.e. ((Gly)₄Ser)ₓ. The characteristics of said peptide linker, which comprise the absence of the promotion of secondary structures are known in the art and described e.g. in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Also particularly preferred peptide linker which comprise less amino acid residues. An envisaged peptide linker with less than 5 amino acids comprises preferably 4, more preferably 3, more preferably 2 and most preferably one amino acids. A particularly preferred “single” amino acid in context of said “peptide linker” is Gly. Accordingly, said peptide linker may consists of the single amino acid Gly. Yet, other amino acids are envisaged. Furthermore, peptide linkers which also do not promote any secondary structures are preferred. As mentioned above, the linker between said first domain and said second domain of an individual monomer comprised in the inventive trimeric polypeptide construct may also be absent.

The linkage of said domains to each other can be provided by, e.g. genetic engineering, as described in the examples. Methods for preparing fused and operatively linked polypeptide chains and expressing them in mammalian cells or bacteria are well-known in the art (e.g. Sambrook et al., Molecular Cloning: A Laboratory Manual, Cold Spring Harbor Laboratory Press, Cold Spring Harbor, N.Y., 1989).

According to a preferred embodiment, the complete extracellular domain of 4-1BBL is employed as “trimerization region” in an individual monomer of the inventive polypeptide construct.

The complete extracellular domain of 4-1BBL comprise the region which is designated as stalk region as well as the TNF homology domain (THD); see FIG. 1. The amino acid sequence of the human 4-1BBL has been described in Goodwin et al. (Eur. J. Immunol. (1993) 23, 2631). In said amino acid sequence the stalk region corresponds to amino acid residues 50 to 91 and the THD corresponds to amino acid residues 92 to 254. Alternatively, it is also preferred that the trimerization region consists only of fragments of the extracellular domain of 4-1BBL which promote the trimerization of the molecule. Said fragments may be optionally linked by suitable additional peptide linker.

Although, no cleavage site has been described for 4.1BBL (Bodmer et al., TIBS (2002) 27(1), 19-26), the introduction of mutations in the stalk domain may be advantageously in order to increase stability in terms of proteolytic degeneration, e.g. proteolytic degeneration during storage at 4° C.

According to a further preferred embodiment the antigen-interaction-site of the trimeric polypeptide construct of the invention comprises at least two domains which specifically interact with separate antigens.

Said preferred embodiment of the invention relates to a trimeric polypeptide constructs which comprise more than one antigen-interaction-site with different specificity. Said embodiment also comprise trimeric polypeptide constructs which comprise at least two domains which specifically interact with separate regions of one molecule which represent individual antigenic determinantes.

More preferably said at least two domains which specifically interact with separate antigens are combined via a peptide linker, wherein said peptide linker does not comprise any polymerization activity.

Peptide linkers are illustrated herein above and exemplified in the appended illustrative examples. Yet, further peptide linkers known in the art may be employed in context of the invention. Also preferred are peptide linkers which comprise repetitive sequence motives of the above described peptide linker as long as said repetitive structure does not comprise any polymerization activity.

Further preferred in accordance with the invention are said antigen-interaction-site of the trimeric polypeptide construct specific for one or more cell surface marker. The term “cell surface marker”, as used herein, denotes molecules which are presented on the surface of cells. Examples for said cell surface marker are membrane and transmembrane proteins, molecules adapted to said proteins or the cell surface etc.

According to a further preferred embodiment of the invention said cell surface marker is a tumor marker.

Examples for said tumor markers are TAG72, PSMA, CD44v6, CEA, Her2-neu, Her-3, Her-4, Lewis Y.

In a preferred embodiment said antigen-interaction-site of the second domain of the monomer of the trimeric polypeptide construct of the invention comprises at least one domain which is an antibody-derived region.

The term “antibody derived region” defines in accordance with the present invention at least one fragment or derivative of an antibody which is characterized by its ability of specific binding and interaction with an epitope. Preferably, said antibody derived region comprises a polypeptide sequence which corresponds to at least one variable region or at least one hypervariable region (CDR) of an antibody.

The term “derived from” means in this context that the region is derived from a domain of an antibody and may comprise substitution(s), deletions(s), addition(s), inversion(s), duplication(s), recombinations, etc.

Furthermore, as defined herein below, “derived from” also envisages derivatives of antibodies like single chain antibodies, preferably scFv or bispecific molecules, like bispecific scFv.

Preferably, one domain which is an antibody-derived region comprises a polypeptide sequence which corresponds to at least two variable regions of an antibody. A particularly preferred molecular format of the invention provides a polypeptide construct wherein the antibody-derived region comprises one V_{H} and one V_{L} region.

The antibody-derived region may be derived from an antibody of any mammalian species. Preferably, said antibody-derived region is derived from a rat, murine or human antibody.

The antibody providing the antigen-interaction-site for the monomer of the trimeric polypeptide construct of the invention can be derived from, e.g., a monoclonal antibody, polyclonal antibody, chimeric antibody, humanized antibody, bispecific antibody, synthetic antibody, antibody fragment or derivative, such as Fab, Fv or scFv fragments etc., or a chemically modified derivative of any of these. Furthermore, antibodies or fragments thereof to the aforementioned antigens can be obtained by using methods which are described, e.g., in Harlow and Lane “Antibodies, A Laboratory Manual”, CSH Press, Cold Spring Harbor, 1988. Antibodies might be obtained from several species, including human. When derivatives of said antibodies are obtained by the phage display technique, surface plasmon resonance as employed in the BIAcore system can be used to increase the efficiency of phage antibodies which bind to an desired epitope (Schier, Human Antibodies Hybridomas 7 (1996), 97-105; Malmborg, J. Immunol. Methods 183 (1995), 7-13). The production of chimeric antibodies is described, for example, in WO 89/09622. Methods for the production of humanized antibodies are described in, e.g., EP-A1 0 239 400 and WO 90/07861. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735.

Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning: A Laboratory Manual, Cold Spring Harbor Laboratory (1989) N.Y. The modification referred to are preferably carried out at the nucleic acid level.

In a further preferred embodiment of the invention said antigen-interaction-site of the second domain of the monomer of the trimeric polypeptide construct comprises at least two antibody-derived regions.

This embodiment comprises e.g. polypeptide constructs which have a specificity of two different antibodies for two different epitopes. Corresponding polypeptide constructs are described in the appended examples.

Particularly preferred are constructs which comprise as the antigen-interaction-site bispecific scFv constructs.

The trimeric polypeptide construct of the invention may be construct, wherein said antigen-interaction-site comprises the extracellular domain of a member of the B7 family or a fragment or a derivative thereof which is capable of binding to its specific receptor.

The B7 family is a group of costimulatory molecules. Examples of members of said family as well as the corresponding specific receptors are described e.g. in Coyle and Gutierrez-Ramos (Nature immunology (2001) 2(3); 203-209).

The term “fragment or a derivative” of a member of the B7 family, as used herein, denotes polypeptides, or secondarily modified polypeptides, derived from the extracellular part of members of the B7 family, which have the capacity to specifically bind to the receptor, to which the member of the B7 family from which they are derived from specifically binds to.

In accordance with the invention the trimeric polypeptide construct is a construct, wherein said antigen-interaction-site is selected from the group consisting of scFv, Fab, and single Ig variable regions.

Preferably it is envisaged that antibody-derived regions which may be the antigen-interaction-site are chimeric antibodies, fully human antibodies or antibodies of non-human-origin which may optionally be humanized, CDR grafted or deimmunized antibodies.

The scFv construct in the antigen-interaction-site may be selected from the group consisting of scFv specific for EpCAM, NKG2D, CD19, PSMA, MCSP, stn (TAG72), CD44v6, carbonic anhydrase IX (CAIX), CEA, EGFR, CD33, Wue-1, CD3, Muc-1, CD20, Her2-neu, Her 3, Her 4 and Lewis-Y.

As mentioned above, it is also preferred that the trimeric polypeptide construct of the invention comprises monomers which are characterized by an antigen-interaction-site which is a bispecific scFv. Accordingly, it is also envisaged that the antigen-interaction-site may bind to/interact with or detect two different antigens. Similarly said antigen-interaction-site may comprise, inter alia, one scFv and a further antigen-interaction-site, like, e.g. a member of the B7 familiy or a fragment of derivative thereof. Corresponding embodiments are illustrated below and in the appended examples.

The above referred member of the B7 family or a fragment or a derivative thereof may be selected from the group consisting of B7.1, B7.2, B7-H3, B7-RP1, B7-DC, PDL1 und PDL2.

The specific receptors for B7.1, B7.2, B7-RP1, B7-DC, PDL1 und PDL2 are disclosed in Coyle and Gutierrez-Ramos (Nature immunology (2001) 2(3); 203-209) and are CD28 (B7.1/B7.2), CTLA-4 (B7.1/B7.2), ICOS (B7RP-1) and PD-1 (PD-L1/PD-L2).

In a particularly preferred embodiment of the invention said second domain of each monomer comprises a scFv specific for EpCAM or ,as illustrated above, several, e.g. two, antigen-interaction-sites, wherein one of said sites is a scFv for EpCAM. The epitope EpCAM has been previously described, e.g. in Raum et al., 2001, Cancer Immunol Immunother. 50(3), 141-150.

In a further preferred embodiment of the invention each monomer which forms the trimeric polypeptide construct has the amino acid sequence as shown in SEQ ID NO: 20.

The monomer having the amino acid sequence as shown in SEQ ID NO: 20 is encoded by a nucleic acid molecule as shown in SEQ ID NO: 19. As described in detail herein below, a preferred embodiment of present invention is directed to nucleic acid molecules encoding said monomers, as well as nucleic acid molecules encoding monomers functionally variants thereof.

In the context of this invention, it is of note, that not only “homo-trimeric constructs” are envisaged, but also “hetero-trimeric constructs”, which are described below. Accordingly, the term “said second domain of each monomer” is not limiting to the trimeric polypeptide constructs which consist of three identical monomers. Accordingly, the monomers as described herein may also be combined to “hetero-trimeric polypeptide constructs” and are envisaged in this invention.

The term “functionally variant” of a monomer of the trimeric polypeptide construct describes in the context of the present invention monomers capable of trimerization as disclosed in detail above and capable of binding to/interaction with a particularly defined antigen via its antigen-interaction site.

Here it was surprisingly found that merely trimererized constructs as described herein are capable of activating an enduring/long lasting T cell response The term “enduring T cell response” means that T cells that have been primed through a TCR- or TCR-like signal plus adequate costimulation. Said T cells prolonged survival e.g. because of protection against activation-induced cell death. As a result, activated T cells are available for prolonged periods of time to act as effector cells on their respective targets. The effect on T cell survival may be analysed by measuring the increase of the expression level of antiapoptotic factors e.g. from the Bcl-2 family like Bclw, Bcl-2, Bcl-xL or Bfl-1 (Jones (2000) J. Exp. Med. 191:1721).

Functional variants show the same specificity of the antigen-interaction-site for the same antigenic epitopes although said functional variants differ in the particular amino acid sequence. Accordingly, said functional variants of the monomer are encoded by nucleic acid sequences having a sequence which is different to the sequence of the nucleic acid molecules to which it is particularly referred to.

In an alternatively particularly preferred embodiment of the invention comprises a scFv of the monoclonal antibody 237.

As known in the art monoclonal antibody detects/interacts with a surface marker of murine sarcoma cell line. The marker is a tumour specific cell surface antigen (PW237 antibody published in Ward et al., 1989, J. Exp. Med. Volume 170, 217-232). A corresponding trimeric polypeptide construct may have the amino acid sequence as shown in SEQ ID NO: 8.

The monomer having the amino acid sequence as shown in SEQ ID NO: 8 may be encoded by a nucleic acid molecule as shown in SEQ ID NO: 7. The invention also comprises nucleic acid molecules encoding said monomers, as well as nucleic acid molecules encoding functional variants of said monomers.

This preferred trimeric polypeptide construct comprising scFv anti-237—murine 4-1BBL, may be used in a mouse model system to quantify the enduring T-cell response triggered. Mice are challenged with tumorigenic cells cultured in vitro and expressing the 237 antigen on their surface. By using the corresponding cells for tumor challenge in a mouse, subsequent injections of scFv anti-237—murine 4-1BBL are performed and the effect on tumor outgrowth can be measured by methods known in the art.

As illustrated in the examples said scFv derived from monoclonal antibody 237 may also be combined with other/another antigen-interaction site(s), like another scFv. Further combinations are also envisaged. Accordingly, the invention also provides for, inter alia, trimeric constructs as defined herein, wherein each or at least one monomer comprises several, preferably two scFvs, like a scFv specific for EpCAM and a scFv specific for NKG2D.

The NKG2D molecule is described in detail in Baur et al. (Science (1999) 285, 727-729).

A corresponding monomer of the trimeric polypeptide construct of the invention may have the amino acid sequence as shown in SEQ ID NO: 18.

The monomer having the amino acid sequence as shown in SEQ ID NO: 18 may be encoded by a nucleic acid molecule as shown in SEQ ID NO: 17. The invention also comprises nucleic acid molecules encoding said monomers, as well as nucleic acid molecules encoding functional variants of said monomers.

As mentioned herein, in a particular preferred embodiment the invention provides for trimeric polypeptide constructs which comprise at least one, preferably two and most preferably three monomers which have as “second domain”, i.e. as antigen-interaction-site, a bispecific construct. Most preferably comprises two scFv's or one scFv and a member of the B7-family (or a part or a fragment thereof). Corresponding examples of such constructs are appended.

In an alternatively particularly preferred embodiment of the invention each monomer comprises a bispecific scFv construct wherein at least one specificity of said bispecific scFv construct is specific for CD3.

In such a monomer said scFv specific for CD3 may have the amino acid sequence as shown in SEQ ID NO: 22.

The monomer having the amino acid sequence as shown in SEQ ID NO: 22 may be encoded by a nucleic acid molecule as shown in SEQ ID NO: 21. The invention also comprises nucleic acid molecules encoding said monomers, as well as nucleic acid molecules encoding functional variants of said monomers.

The second domain of the monomer of the inventive trimeric construct may also comprise a scFv specific for EpCAM and a antigen-interaction side which is the extracellular domain of B7.1 or a fragment or a derivative thereof which is capable of binding to its specific receptor, namely CD28 or CTLA-4.

Such a construct monomer may have the amino acid sequence as shown in SEQ ID NO: 16 and may be encoded by a nucleic acid molecule as shown in SEQ ID NO: 15. The invention also comprises nucleic acid molecules encoding said monomers, as well as nucleic acid molecules encoding functional variants of said monomers.

In accordance with the invention it is envisaged that the trimeric polypeptide construct is consisting of at least two different monomers, wherein said different monomers are characterized by different antigen-interaction-sites. As pointed out above, the term “second domain of each monomer” is not limiting to trimeric polypeptide constructs which comprise identical monomers, i.e. “homo-trimeric constructs”. Also envisaged are “hetero-trimeric constructs”, wherein at least one monomer differs from the other monomer(s) comprised in said trimeric polypeptide construct. It is, e.g., possible that the three monomers comprise different antigen-interaction sites and/or that only one monomer comprises an additional tag or label, like a HIS tag.

Accordingly, the present invention also provides for trimeric polypeptide constructs which are formed by two or three different monomers. Such constructs are considered as heterotrimers or hetero-trimeric constructs. It is most preferred that the heterotrimers comprise monomers which have or comprise a first domain which promotes the trimerization of said monomer, wherein said first domains of said three monomers are preferably identical. Yet, in a prffferred embodiment of the heterotrimeric constructs of the invention, different second domains, i.e. antigen-interaction-sites, are envisaged in the individual monomers of the trimeric polypeptide construct.

Further, according to said embodiment the two or three different monomers can be distinguished by there different second domains. Said second domains may consist of one ore more antigen-interaction sites with specificity for different antigens or antigenic determents of one or more molecules.

Preferably, the heterotrimeric polypeptide construct of the invention may consist of at least one monomer which has an antigen-interaction sites with specificity for an target cell antigen and at least one monomer which has an antigen-interaction sites with specificity for an activation molecule on an effector cell.

It is envisaged that at least one monomer of the trimeric polypeptide construct further comprises a tag.

The term “tag” is known in by the person skilled in the art and designates a label, by which a polypeptide comprising a tag may be identified.

Said tag may be selected from the group consisting of: His-tag, Flag-tag, Myc-tag, HA-tag, GST-tag, T100™, VSV-G, V5, S-tag™, HSV, CFP, RFP, YFP, GFP, BFP, Cellulose binding domain (CBD), Maltose binding protein (MBP), NusA-tag, thioredoxin (Trx), DsbA, DabC and a biotinylation sequence.

Most preferably, said tag is a His-tag at the C-terminus of the at least one monomer.

As mentioned above the trimeric polypeptide construct of the invention is preferably expressed in a eukaryotic expression system.

Eukaryotic expression systems are described herein below in detail.

The present invention also provides for a nucleic acid molecule encoding a monomer of a trimeric polypeptide construct of the invention.

Thus, the present invention relates to a nucleic acid molecule comprising a nucleotide sequence selected form the group consisting of:

(a) a nucleotide sequence encoding the mature form of a protein comprising the amino acid sequence of a monomer of a trimeric polypeptide construct of the invention, preferably as given in SEQ ID Nos: 20, 8, 18, 22 and 16;

(b) a nucleotide sequence comprising or consisting of the DNA sequence as given in SEQ ID Nos: 19, 7, 17, 21 and 15;

(c) a nucleotide sequence hybridizing with the complementary strand of a nucleotide sequence as defined in (b) under stringent hybridization conditions;

(d) a nucleotide sequence encoding a protein derived from the protein encoded by a nucleotide sequence of (a) or (b) by way of substitution, deletion and/or addition of one or several amino acids of the amino acid sequence encoded by the nucleotide sequence of (a) or (b);

(e) a nucleotide sequence encoding a protein having an amino acid sequence at least 60% identical to the amino acid sequence encoded by the nucleotide sequence of (a) or (b);

(f) a nucleotide sequence which is degenerate as a result of the genetic code to a nucleotide sequence of any one of (a) to (e);

The term “mature form of the protein” defines in context with the present invention a protein translated from its corresponding mRNA and optional subsequently modified.

The term “hybridizing” as used herein refers to polynucleotides which are capable of hybridizing to the polynucleotides of the invention or parts thereof. Therefore, said polynucleotides may be useful as probes in Northern or Southern Blot analysis of RNA or DNA preparations, respectively, or can be used as oligonucleotide primers in PCR analysis dependent on their respective size. Preferably, said hybridizing polynucleotides comprise at least 10, more preferably at least 15 nucleotides in length while a hybridizing polynucleotide of the present invention to be used as a probe preferably comprises at least 100, more preferably at least 200, or most preferably at least 500 nucleotides in length.

It is well known in the art how to perform hybridization experiments with nucleic acid molecules, i.e. the person skilled in the art knows what hybridization conditions s/he has to use in accordance with the present invention. Such hybridization conditions are referred to in standard text books such as Molecular Cloning A Laboratory Manual, Cold Spring Harbor Laboratory (1989) N.Y. Preferred in accordance with the present inventions are polynucleotides which are capable of hybridizing to the polynucleotides of the invention or parts thereof, under stringent hybridization conditions.

“Stringent hybridization conditions” refer, i.e. to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. Also contemplated are nucleic acid molecules that hybridize to the polynucleotides of the invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6× SSPE (20×SSPE=3 M NaCl; 0.2 M NaH₂PO4; 0.02 M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). It is of note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Nucleic acid molecule of the invention may be, e.g., DNA, cDNA, RNA or synthetically produced DNA or RNA or a recombinantly produced chimeric nucleic acid molecule comprising any of those polynucleotides either alone or in combination.

The invention also provides for a vector comprising an above defined nucleic acid molecule of the invention.

Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley lnterscience, N.Y. (1989), (1994). Alternatively, the polynucleotides and vectors of the invention can be reconstituted into liposomes for delivery to target cells. As discussed in further details below, a cloning vector was used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

The nucleic acid molecule comprised in said vector is DNA.

It is envisaged that the vector of the invention is an expression vector wherein the nucleic acid molecule encoding a monomer of a trimeric polypeptide construct of the invention is operatively linked to one or more control sequences allowing the transcription and optionally expression in prokaryotic and/or eukaryotic hosts.

The term “control sequence” refers to regulatory DNA sequences which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used. Thus, the vector of the invention is preferably an expression vector. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the P_{L}, lac, trp or tac promoter in E. coli, and examples of regulatory elements permitting expression in eukaryotic host cells are the AOX1 or GAL 1 promoter in yeast or the CMV-, SV40-, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art; see also, e.g., the appended examples. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 l (Pharmacia), pCDM8, pRc/CMV, pcDNA1, pcDNA3 (In-vitrogene), pEF-DHFR and pEF-ADA (Raum et al. Cancer Immunol Immunother (2001) 50(3), 141-150) or pSPORT1 (GIBCO BRL).

Preferably, the expression control sequences will be eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the invention may follow; see, e.g., the appended examples.

An alternative expression system which could be used to express a cell cycle interacting protein is an insect system. In one such system, Autographa californica nuclear polyhedrosis virus (AcNPV) is used as a vector to express foreign genes in Spodoptera frugiperda cells or in Trichoplusia larvae. The coding sequence of a nucleic acid molecule of the invention may be cloned into a nonessential region of the virus, such as the polyhedrin gene, and placed under control of the polyhedrin promoter. Successful insertion of said coding sequence will render the polyhedrin gene inactive and produce recombinant virus lacking coat protein coat. The recombinant viruses are then used to infect S. frugiperda cells or Trichoplusia larvae in which the protein of the invention is expressed (Smith, J. Virol. 46 (1983), 584; Engelhard, Proc. Nat. Acad. Sci. USA 91 (1994), 3224-3227).

Additional regulatory elements may include transcriptional as well as translational enhancers. Advantageously, the above-described vectors of the invention comprises a selectable and/or scorable marker.

Selectable marker genes useful for the selection of transformed cells and, e.g., plant tissue and plants are well known to those skilled in the art and comprise, for example, antimetabolite resistance as the basis of selection for dhfr, which confers resistance to methotrexate (Reiss, Plant Physiol. (Life Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Nati. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable marker are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a vector of the invention.

As described above, the nucleic acid molecule of the invention can be used alone or as part of a vector to express the polypeptide of the invention in cells, for, e.g., gene therapy. The nucleic acid molecules or vectors containing the DNA sequence(s) encoding any one of the above described trimeric polypeptide constructs is introduced into the cells which in turn produce the polypeptide of interest. Gene therapy, which is based on introducing therapeutic genes into cells by ex-vivo or in-vivo techniques is one of the most important applications of gene transfer. Suitable vectors, methods or gene-delivery systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91 (1998), 30-36; Verma, Gene Ther. 5 (1998), 692-699; Nabel, Ann. N.Y. Acad. Sci. 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; U.S. Pat. No. 5,589,466; or Schaper, Current Opinion in Biotechnology 7 (1996), 635-640. The nucleic acid molecules and vectors of the invention may be designed for direct introduction or for introduction via liposomes, or viral vectors (e.g., adenoviral, retroviral) into the cell. Preferably, said cell is a germ line cell, embryonic cell, or egg cell or derived therefrom, most preferably said cell is a stem cell. An example for an embryonic stem cell can be, inter alia, a stem cell as described in, Nagy, Proc. Natl. Acad. Sci. USA 90 (1993), 8424-8428.

In accordance with the above, the present invention relates to vectors, particularly plasmids, cosmids, viruses and bacteriophages used conventionally in genetic engineering that comprise a nucleic acid molecule encoding a monomer of a trimeric polypeptide construct of the invention. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell populations. Methods which are well known to those skilled in the art can be used to construct recombinant vectors; see, for example, the techniques described in Sambrook, Molecular Cloning: A Laboratory Manual, Cold Spring Harbor Laboratory (1989) N.Y. and Ausubel, Current Protocols in Molecular Biology, Green Publishing Associates and Wiley lnterscience, N.Y. (1989). Alternatively, the nucleic acid molecules and vectors of the invention can be reconstituted into liposomes for delivery to target cells. The vectors containing the nucleic acid molecules of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

The vector of the invention may be the pEF-DHFR or pEF-ADA.

The vectors pEF-DHFR and pEF-ADA have been described in the art, e.g. in Mack et al. (PNAS (1995) 92, 7021-7025) and Raum et al. (Cancer Immunol Immunother (2001) 50(3), 141-150).

The present invention furthermore relates to host containing at least one vector or at least one nucleic acid molecule of the invention.

Said host may be produced by introducing said at least one vector or at least one nucleic acid molecule into the host. The presence of said at least one vector or at least one nucleic acid molecule in the host may mediate the expression of a gene encoding a monomer of the trimeric polypeptide construct of the invention.

The nucleic acid molecule or vector of the invention which is present in the host may either be integrated into the genome of the host or it may be maintained extrachromosomally.

The host can be any prokaryote or eukaryotic cell.

The term “prokaryote” is meant to include all bacteria which can be transformed or transfected with a DNA or RNA molecules for the expression of a protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the protein encoded by the polynucleotide of the present invention may be glycosylated or may be non-glycosylated. Especially preferred is the use of a plasmid or a virus containing the coding sequence of the polypeptide of the invention and genetically fused thereto an N-terminal FLAG-tag and/or C-terminal His-tag. Preferably, the length of said FLAG-tag is about 4 to 8 amino acids, most preferably 8 amino acids. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989).

In a preferred embodiment the host is a bacteria, an insect, fungal, plant or animal cell.

Preferably it is envisaged that the host of the invention may be a mammalian cell, more preferably a human cell or human cell line

Particularly preferred host cells comprise CHO cells, COS cells, myeloma cell lines like SP2/0 or NS/0 .

An alternative embodiment of the invention relates to a process for the production of a trimeric polypeptide construct of the invention, said process comprising culturing a host of the invention under conditions allowing the expression of the polypeptide construct and recovering the produced polypeptide construct from the culture.

The transformed hosts can be grown in fermentors and cultured according to techniques known in the art to achieve optimal cell growth. The polypeptide of the invention can then be isolated from the growth medium, cellular lysates, or cellular membrane fractions. The isolation and purification of the, e.g., microbially expressed polypeptides of the invention may be by any conventional means such as, for example, preparative chromatographic separations and immunological separations such as those involving the use of monoclonal or polyclonal antibodies directed, e.g., against a tag of the polypeptide of the invention or as described in the appended examples.

The conditions for the culturing of a host which allow the expression are known in the art to depend on the host system and the expression system/vector used in such process. The parameters to be modified in order to achieve conditions allowing the expression of a recombinant polypeptide are known in the art. Thus, suitable conditions can be determined by the person skilled in the art in the absence of further inventive input.

Once expressed, the polypeptide constructs of the present invention can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure polypeptides of at least about 90 to 95% homogeneity are preferred, and 98 to 99% or more homogeneity are most preferred, for pharmaceutical uses. Once purified, partially or to homogeneity as desired, the polypeptides may then be used therapeutically (including extracorporeally) or in developing and performing assay procedures. Furthermore, examples for methods for the recovery trimeric polypeptide construct of the invention from a culture are described in detail in the appended examples.

Preferably, the expression in the process of the invention leads to an rate of trimerization of at least 90% and recovering the produced polypeptide construct from the culture. More preferably the rate of trimerization of at least 95%, most preferably of at least 99%.

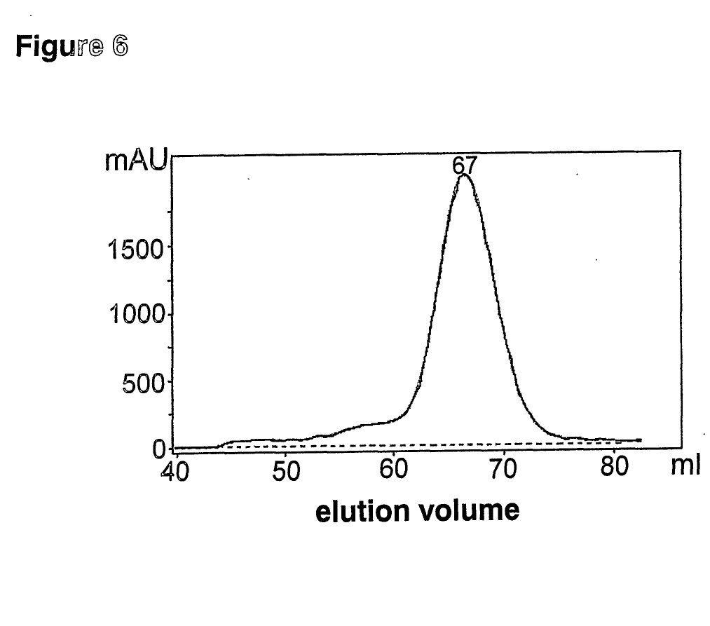

Methods to determine the rate of trimerization of a polypeptide are known in the art. One example of a suitable method is particularly described in appended example 1 and the result of such determination is depicted in FIG. 6.

to the invention also provides for a composition comprising a trimeric polypeptide construct of the invention, a trimeric polypeptide construct as produced by the process of the invention, a nucleic acid molecule of the invention, a vector of the invention or a host of the invention and, optionally, a proteinaceous compound capable of providing an activation signal for immune effector cells.

In the light of the present invention, said “proteinaceous compounds” providing an activation signal for immune effector cells” may be, e.g. a primary activation signal for T cells. Preferred formats of proteinaceous compounds comprise bispecifc antibodies and fragments or derivatives thereof, e.g. bispecific scFv. Preferably, said primary activation signal for T cells may be provided via the T cell receptor (TCR), more preferably via CD3 molecule of the TCR. Proteinaceous compounds can comprise, but are not limited to, scFv fragments specific for CD3, scFv fragments specific for the T cell receptor or superantigens. Superantigens directly bind to certain subfamilies of T cell receptor variable regions in an MHC-independent manner thus mediating the primary T cell activation signal. The proteinaceous compound may also provide an activation signal for immune effector cell which is a non-T cell. Examples for immune effector cells which are non-T cells comprise, inter alia, B cells and NK cells.

The present invention also relates to compositions which are pharmaceutical compositions comprising these aforementioned trimeric polypeptide construct(s), nucleic acid molecule(s), vector(s) or host(s) of the invention and, optionally, the described proteinaceous compound capable of an activation signal for immune effector cells.

The compositions of the invention, which are pharmaceutical compositions may be administered simultaneous or in a non-simultaneous way with an above defined proteinaceous compound capable of an activation signal for immune effector cells.

In a further preferred embodiment of the invention the composition, which is a pharmaceutical composition, further comprises suitable formulations of carrier, stabilizers and/or excipients.

Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regiment will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. However, a more preferred dosage for continuous infusion might be in the range of 0.01 μg to 10 mg units per kilogram of body weight per hour. Particularly preferred dosages are recited herein below. Progress can be monitored by periodic assessment. Dosages will vary but a preferred dosage for intravenous administration of DNA is from approximately 10⁶ to 10¹² copies of the DNA molecule. The compositions of the invention may be administered locally or systematically. Administration will generally be parenterally, e.g., intravenously; DNA may also be administered directed to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present invention might comprise proteinaceous carriers, like, e.g., serum albumine or immunoglobuline, preferably of human origin. Furthermore, it is envisaged that the pharmaceutical composition of the invention might comprise further biologically active agents, depending on the intended use of the pharmaceutical composition. Such agents might be drugs acting on the gastro-intestinal system, drugs acting as cytostatica, drugs preventing hyperurikemia and/or agents such as T-cell co-stimulatory molecules or cytokines known in the art.

Possible indications for administration of the trimeric constructs of the invention are tumorous diseases especially epithelial cancers/carcinomas such as mamma carcinom, colon carcinom, prostate carcinom, ovarial carcinom or lung carcinom or other tumorous diseases like haematological tumors, gliom, sarcom or osteosarcom. The administration of the constructs of the invention is especially indicated for minimal residual disease, which is characterized by the local and non-local reoccurrance of the tumor caused by the survival of single cells. The problem with conventional treatments for minimal residual disease such as adjuvant chemotherapy is that only dividing cells are eliminated. Therefore, single tumourous cells may survive chemotherapy in a resting/anergic state and later on may form newly growing tumor. Further possible indications for administration of the constructs of the invention may comprise autoimmune diseases, especially T cell mediated autoimmune diseases, inflammatory diseases (antigen-specific T cell activation), infectious diseases, especially bacterial and fungal infections, viral diseases (treatment long term vaccine), allergic reactions, parasitic reactions, graft versus host disease, transplant rejection.

The invention further envisages the co-administration protocols with other compounds, e.g. bispecific antibody constructs, targeted toxins or other compounds, which act via T cells. The clinical regimen for co-administration of the inventive compound(s) may encompass co-administration at the same time, before or after the administration of the other component.

The trimeric constructs of the invention may also be modified or derivatized. Corresponding modifications may comprise the use of recombinant DNA technologies to improve the binding specificity, avidity, half life etc of the inventive constructs or their monomers. It is also envisaged to reduce the potential residual antigenicity of the constructs.

A possible approach to demonstrate the efficacy/activity of the inventive constructs in an in vivo model like mouse. A suitable model may be the Ag104A (osteosarcoma) mouse model (the cell line was described in Wick et al. J. Exp. Med. 186 (2), Jul.21, 1997 229-238). The Ag104A is a murine fibrosarcoma cell line displaying a tumour specific cell surface antigen (PW237 antibody published in Ward et al., 1989, J. Exp. Med. Volume 170, 217-232). This mouse model could be used to test in vivo tumor regression of transfected Ag104A cells. Such an experiment would be designed to challenge C3H/HeN MMTV⁻ mice with subcutaneous injections in the back.

As detailed herein, the pharmaceutical composition of the invention may be administered to a patient in need of medical intervention (preferably a human patient). The pharmaceutical composition may administered alone or in combination with other medicaments/pharmaceutical compositions. These further medicaments/pharmaceutical compositions may be administered simultaneously or non-simultaneously with the pharmaceutical composition of the invention.

Alternatively the present invention relates in a preferred embodiment to a composition, which is a diagnostic composition further comprising, optionally, means and methods for detection.

A further alternative embodiment of the invention relates to the use of a trimeric polypeptide construct of the invention or as produced by a process of the invention, a nucleic acid molecule of the invention, a vector of the invention or a host of the invention for the preparation of a pharmaceutical composition for the prevention, treatment or amelioration of a proliferative disease, a tumorous disease, an inflammatory disease, an immunological disorder, an autoimmune disease, an infectious disease, viral disease, allergic reactions, parasitic reactions, graft-versus-host diseases or host-versus-graft diseases.

Further preferred said tumorous disease is epithelial cancer or a minimal residual cancer.

It is envisaged by the present invention that the various trimeric polypeptide constructs, nucleic acid molecules and vectors of the invention are administered either alone or in any combination using standard vectors and/or gene delivery systems, and optionally together with a pharmaceutically acceptable carrier or excipient. Subsequent to administration, said nucleic acid molecules or vectors may be stably integrated into the genome of the subject.

On the other hand, viral vectors may be used which are specific for certain cells or tissues and persist in said cells. Suitable pharmaceutical carriers and excipients are well known in the art. The pharmaceutical compositions prepared according to the invention can be used for the prevention or treatment or delaying the above identified diseases.

Furthermore, it is possible to use a pharmaceutical composition of the invention which comprises nucleic acid molecules or vectors of the invention in gene therapy. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Further methods for the delivery of nucleic acids comprise particle-mediated gene transfer as, e.g., described in Verma, Gene Ther.15 (1998), 692-699.

Furthermore the invention relates to a method for the prevention, treatment or amelioration of a proliferative disease, a tumorous disease, an inflammatory disease, an immunological disorder, an autoimmune disease, an infectious disease, viral disease, allergic reactions, parasitic reactions, graft-versus-host diseases or host-versus-graft diseases comprising the step of administering to a subject in need of such a prevention, treatment or amelioration a trimeric polypeptide construct of the invention or as produced by a process of the invention, a nucleic acid molecule of the invention, a vector of the invention or a host of the invention.

Preferably, said tumorous disease is epithelial cancer or a minimal residual cancer. Such epithelial cancers are e.g. mamma carcinoma and other adenocarcinomas, which may be characterized by overexpression of the following cell surface molecules: Her-2 (Arteaga, Semin Oncol 2002 Jun;29(3 Suppl 11):4-10; Wester, Acta Oncol 2002;41 (3):282-8); EpCAM (Naundorf, Int J Cancer 2002 Jul 1;1 00(1):101-10), EGFR (Liu, Br J Cancer 2000 Jun;82(12):1991-9), CEA (Stewart, Cancer Immunol Immunother 1999 Feb;47(6):299-306; Durbin, Proc Natl Acad Sci U S A 1994 May 10;91(10):4313-7), TAG-72 (tumor associated glycoprotein=>sTn antigen) (Kashmiri, Crit Rev Oncol Hematol 2001 Apr;38(1):3-16), MUC-1 (mucin) (Couto, Adv Exp Med Biol 1994;353:55-9), Sonic Hedgehog (Shh) (Lacour, Br J Dermatol 2002 Apr;146 Suppl 61:17-9; Tojo, Br J Dermatol 2002 Jan;146(1):69-73). Further epithelial cancers are squamous cell carcinoma like head and neck cancer, which may be characterized by the overexpression of the following molecules: EGFR (Bonner, Semin Radiat Oncol 2002 July; 12: 11-20; Kiyota, Oncology 2002; 63 (1): 92-8), CD44v6 (Rodrigo, Am J Clin Pathol 2002 Jul;118(1):67-72; Fonseca, J Surg Oncol 2001 Feb;76(2):115-20), prostate cancer, which may be characterized by overexpression of: PSMA (Fracasso, Prostate 2002 Sep 15;53(1):9-23), STEAP (Hubert, Proc Natl Acad Sci U S A 1999 Dec 7;96(25):14523-8), PSCA (prostate stem cell antigen) (Jalkut, Curr Opin Urol 2002 Sep;12(5):401-6). SCLC (small cell lung cancer), which may be characterized by overexpression of ganglioside GD3 (Brezicka, Lung Cancer 2000 Apr;28(1):29-36; Sheperd, Semin Oncol 2001 Apr;28(2 Suppl 4):30-7), ovarian cancer, which may be characterized by mesothelin expression (Scholler, Proc Natl Acad Sci U S A 1999 Sep 28;96(20):11531-6; Brinkmann, Int J Cancer 1997 May 16;71(4):638-44), CA-125 (Hogdall, Anticancer Res 2002 May-Jun;22(3):1765-8), Muellerian Inhibitory Substance (MIS) Receptor Type II (Stephen, Clin Cancer Res 2002 Aug;8(8):2640-6), gastric cancer, which may be characterized by the expression of E-cadherin neoepitope (Becker, Surg Oncol 2000 Jul;9(1):5-1 1), colon carcinoma, which may be characterized by the expression of Lewis-Y (Flieger, Clin Exp Immunol 2001 Jan;123(1):9-14; Power, Cancer Immunol Immunother 2001 Jul;50(5):241-50), A33 antigen (Heath, Proc Natl Acad Sci U S A 1997 Jan 21;94(2):469-74), renal cell carcinoma, which may be characterized by the expression of carbonic anhydrase IX (MN/CA IX) (Uemura, Br J Cancer 1999 Oct;81 (4):741 -6), cervix carcinoma, which may be characterized by the expression of carbonic anhydrase IX (MN/CA IX) (Longcaster, Cancer Res 2001 Sep 1;61(17):6394-9), pancreas carcinoma, which may be characterized by the expression of CA19-9 marker (Brockmann, Anticancer Res 2000 Nov-Dec; 20(6D):4941-7). Furthermore there are numerous epithelial cancers, which are characterized by the expression of Lewis-Y (Power, Cancer Immunol Immunother 2001 Jul;50(5):241 -50).

A minimal residual disease, which is prevented, treated or ameliorated by the present invention is e.g. metastatic disease, which may be characterized by the expression of CD44v6 (Rodrigo, Am J Clin Pathol 2002 Jul;118(1):67-72; Fonseca, J Surg Oncol 2001 Feb;76(2):115-20).

Also preferred is, that the subject to which is referred to is a human.

The method for the prevention, treatment or amelioration of the invention may comprise the co-administration of an above defined proteinaceous compound capable of an activation signal for immune effector cells to the subject. The co-administration may be a simultaneous co-administration or a non-simultaneous co-administration.

Finally, the present invention relates to a kit comprising a trimeric polypeptide construct of the invention or as produced by a process of the invention, a nucleic acid molecule of the invention, a vector of the invention or a host of the invention. It is also envisaged that the kit of this invention comprises a pharmaceutical composition as described herin above, either alone or in combination with further medicaments to be administered to a patient in need of medical treatment or intervention.

The Figures show:

FIG. 1. Schematic of the 4-1BBL structure in comparison to the structure of TNF α precursor protein.

ECD=extracellular domain; THD=TNF homology domain; stalk=stalk-region; aa=amino acid. The arrows indicates the proteolytic cleavage site of tumor necrosis factor α (TNFα) converting enzyme (TACE). The dotted lines point towards individual amino acid positions, the zick-zack-lines represent transmembrane domains.

FIG. 2. Phylogenetic tree analysis. (A) The Treetop program calculates the pairwise distance between the sequences and gives a “bootstrap” value, which hints towards the reproducibility of the tree. 100 is maximum value. Any smaller values indicate the percentage of reproducibility. The topological algorithm uses the topological similarita principle (Chumakov & Yushmanov, 1988, Mol Genet Microbiol Virusol 3, 3-9; Yushmanov & Chumakov, 1988, Mol Genet Microbiol Virusol 3, 9-15; Brodsky et al., 1992, Dimacs 8, 127-139; Brodsky et al., 1995, Biochemistry, 923-928). (B) The unrooted tree.

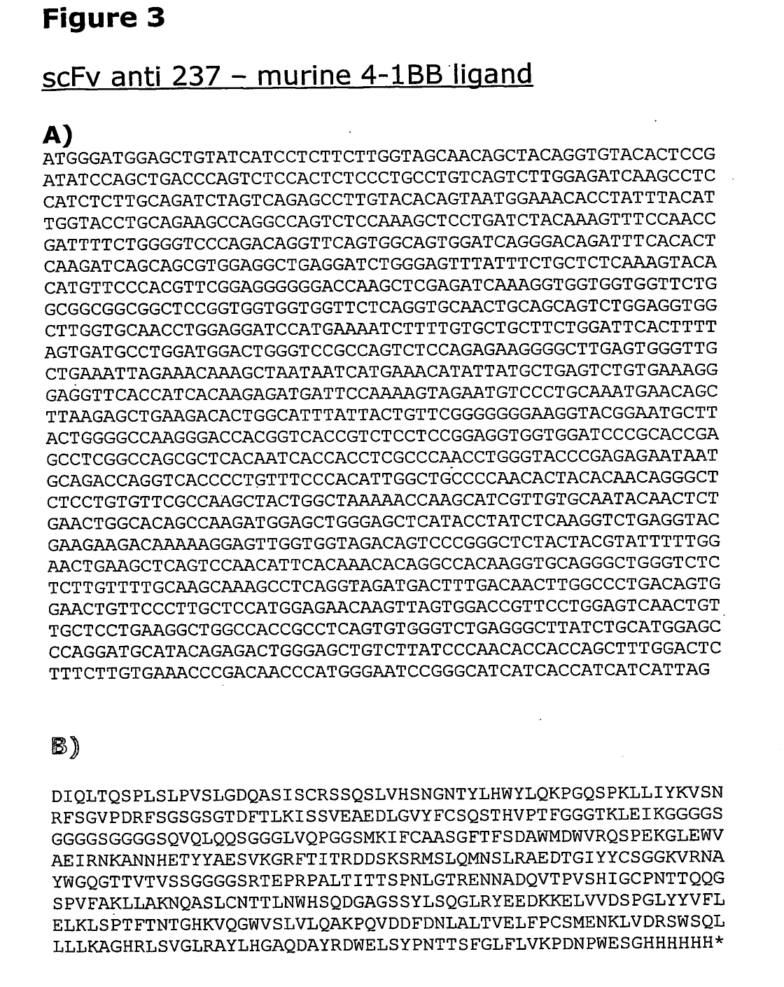

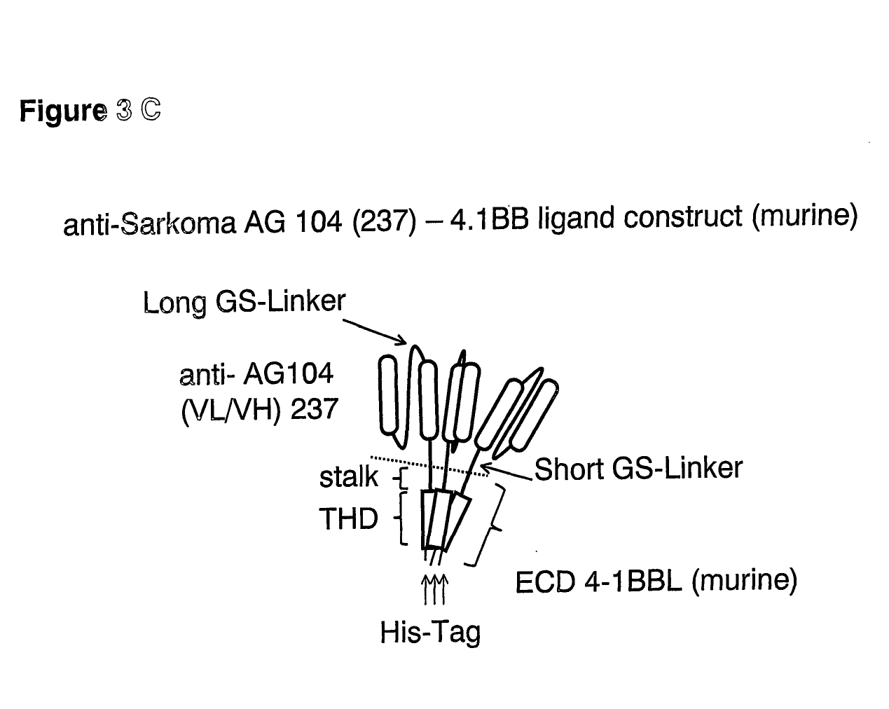

FIG. 3. Sequences scFv anti-237×murine4-1BB ligand construct. A) Nucleotide sequence, B) protein sequence, C) schematic representation of the construct.

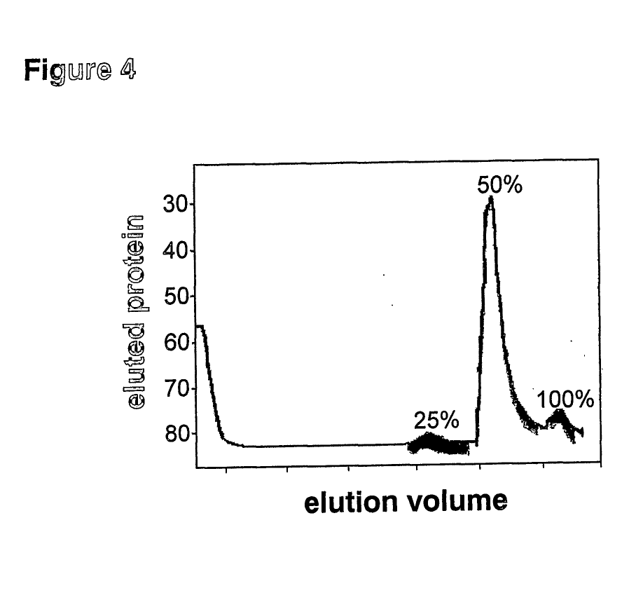

FIG. 4. Elution pattern of scFv 237×murine4.1.BBL from an SP Sepharose cation exchange column. The protein peak from elution with 50% buffer B1 was used for further purification.

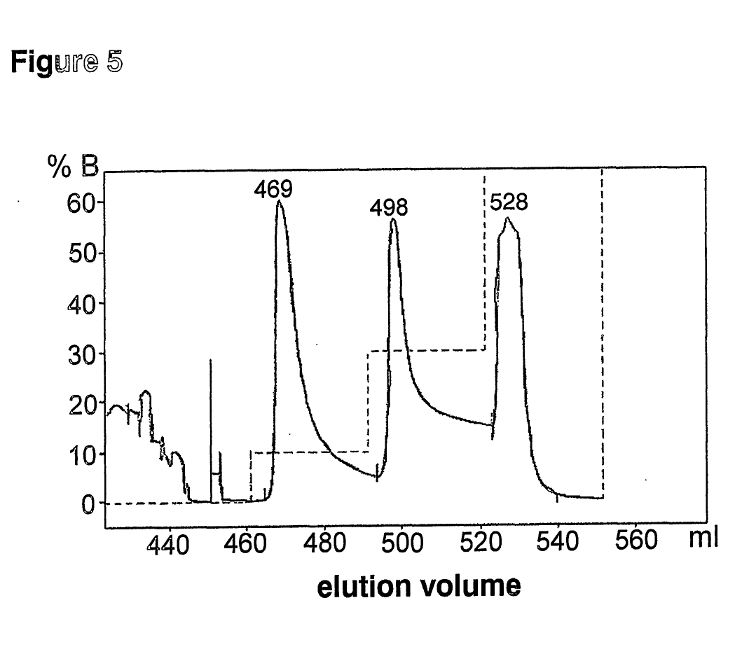

FIG. 5. Elution pattern of scFv anti-237×murine4-1BBL containing protein fractions from a Ni-Chelating His Trap column. The green line indicates the theoretical gradient of elution buffer containing 0.5 M Imidazol. Protein fractions from the 100% buffer B2 elution step were used for further purification.

FIG. 6. Protein elution pattern (blue line) of scFv anti-237×murine4-1BBL construct from a Sephadex S200 gelfiltration column. The protein elutes in a single peak at ca. 67 ml and corresponds to a MW of ca.150 kD. A slight shoulder of the peak with a higher molecular weight can be observed at ca. 58 ml. The monomer elutes in a protein peak at 83.2 ml and corresponds to a MW of ca.54 kD.

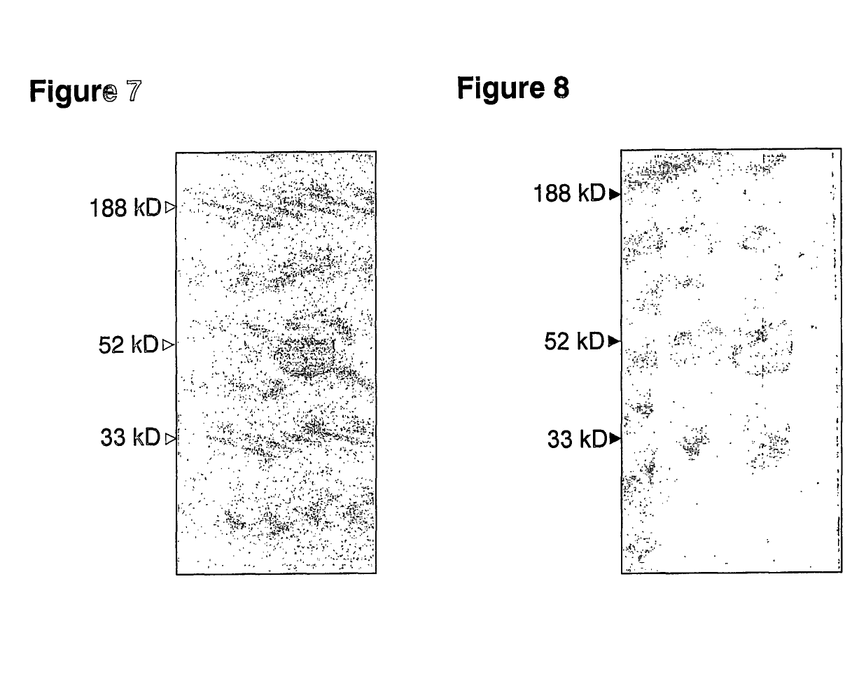

FIG. 7. SDS-PAGE analysis of purified scFv anti-237×murine4-1BBL containing protein fractions. SDS-PAGE was stained with colloidal Coomassie. Lane 1: MultiMark molecular weight marker; lane 2 and 3: gelfiltration fractions of the main peak and the shoulder.

FIG. 8. Western blot analysis of purified scFv anti-237×murine4-1BBL protein fractions. The Western blot was incubated with Penta His antibody and goat anti-mouse antibody labeled with alkaline phosphatase. The stainer was BCIP/NBT liquid. Lane 1: Molecular weight marker; lane 2 and 3: gelfiltration fractions of the main peak and the shoulder. The main band at ca.50 kD contains>90% of the purified protein. The minor band at ca. 100 kD corresponds to a dimeric form of the 237scFv×4.1.BBL and is due to the overloaded gel. The minor band at 33 kD is a proteolytic cleaved fragment.

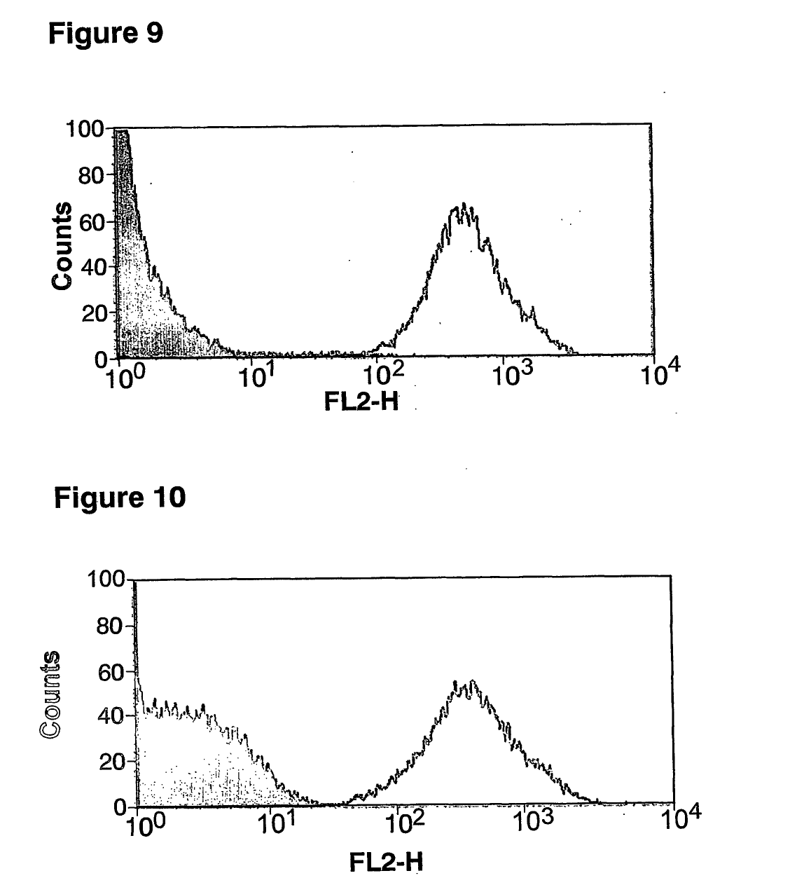

FIG. 9. FACS binding-analysis of the scFv anti-237×murine4-1BBL construct to the AG104A cell line. The FACS staining was performed as described in Example 1 paragraph 4. The filled histogram represents cells incubated with the anti-his antibody and the second step reagent alone. The open histogram shows cells incubated with the construct, the anti-his antibody and the second step antibody.

FIG. 10. FACS analysis of the mu4-1BB ligand portion of the scFv anti-237×murine4-1BBL construct bound to AG104 A cells. The FACS staining was performed as described in Example 1 paragraph 5. The filled histogram represents cells incubated with the anti 4-1BB ligand antibody and the second step reagent alone. The open histogram shows cells incubated with the construct, the anti 4-1BB ligand antibody and the second step reagent.

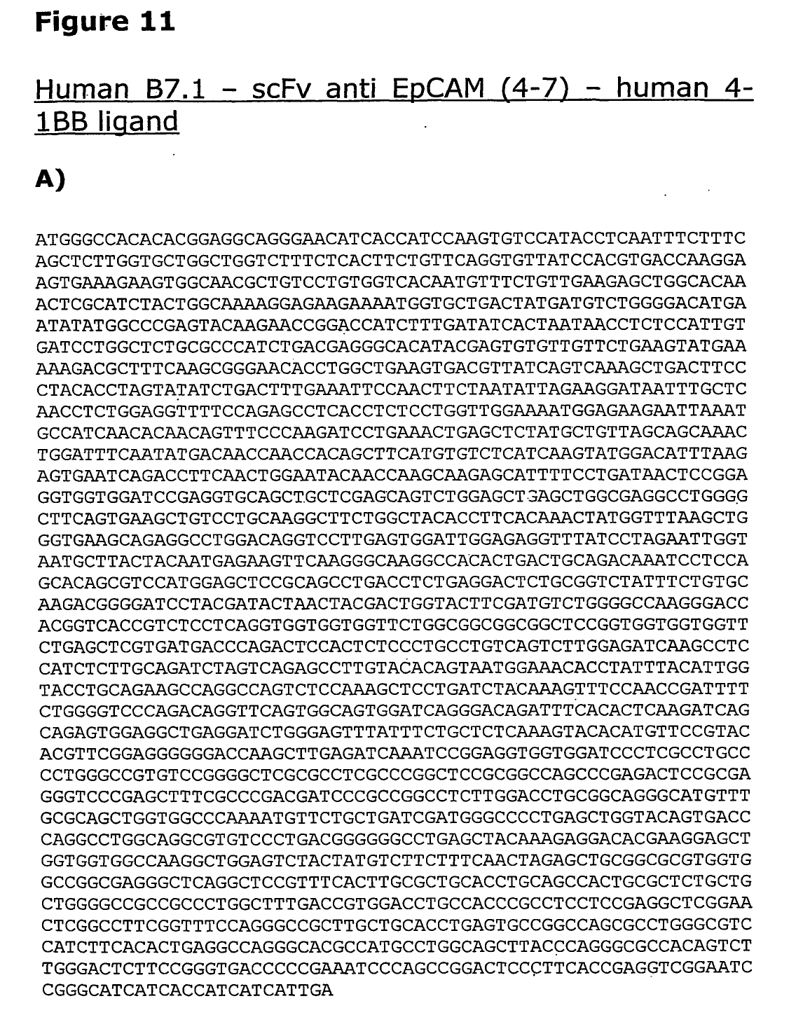

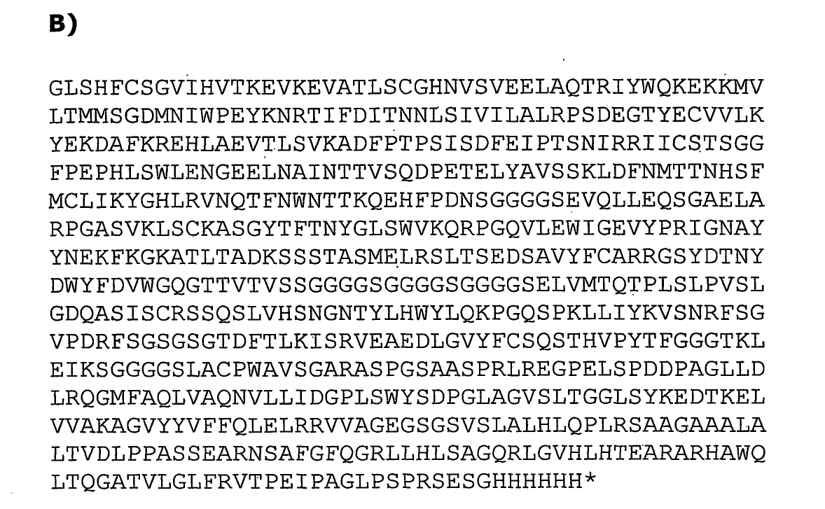

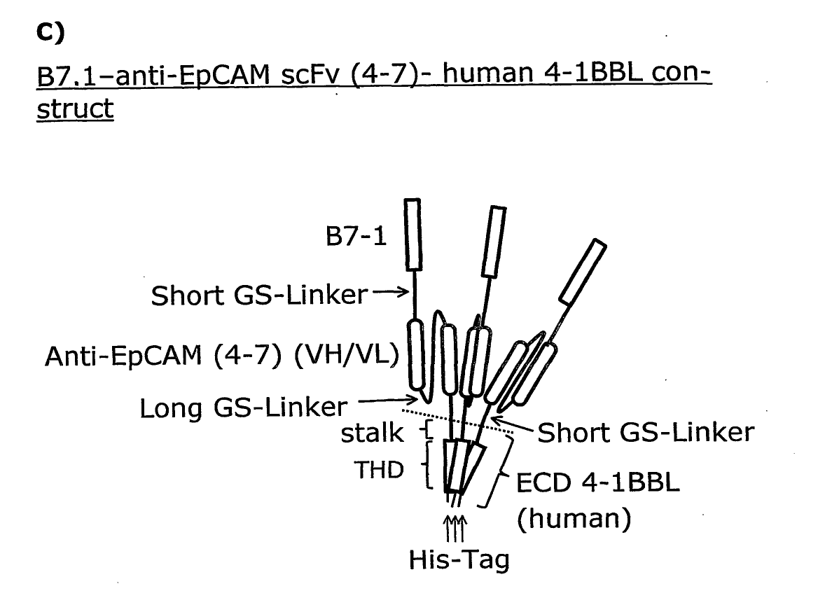

FIG. 11. Sequences of the B7.1—scFv anti-EpCAM (4-7)—human 4-1BB ligand construct. A) Nucleotide sequence, B) protein sequence, C) schematic representation of the construct.

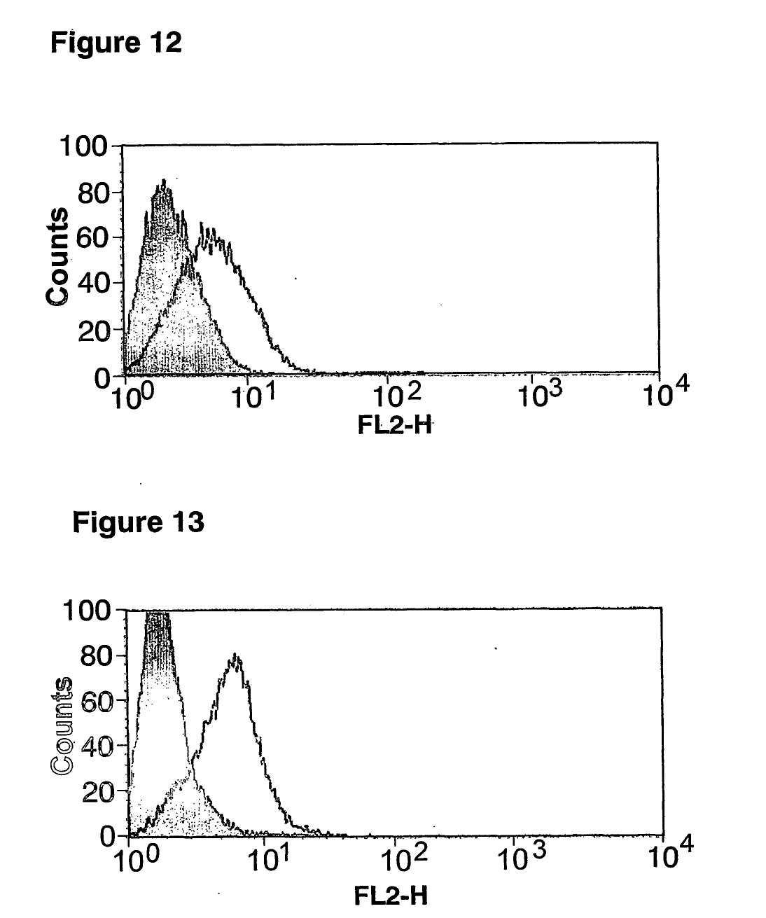

FIG. 12. FACS binding analysis of the B7.1—scFv anti-EpCAM (4-7)—human 4.1BBL construct to the EpCAM antigen on Kato III cells. The FACS staining was performed as described in Example 1A paragraph 4. The filled histogram represents cells incubated with the anti-his antibody and the second step reagent alone. The open histogram shows cells incubated with the construct, the anti-his antibody and the second step antibody.

FIG. 13. FACS analysis of the 4-1BB ligand portion of the B7.1—scFv anti-EpCAM (4-7)—human 4.1BBL construct bound to Kato III cells. The FACS staining was performed as described in Example 1A paragraph 5. The filled histogram represents cells incubated with the anti 4-1BB ligand antibody alone. The open histogram shows cells incubated with the construct and the anti 4-1BB ligand antibody.

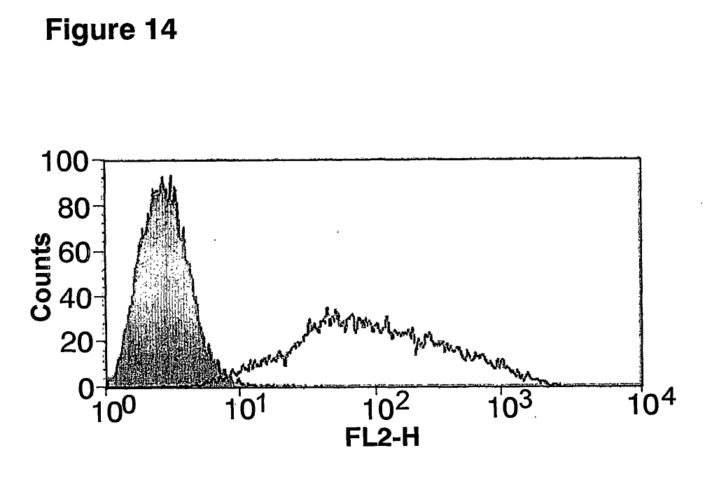

FIG. 14. FACS analysis of the B7.1 portion of the B7.1—scFv anti-EpCAM (4-7)—human 4.1BBL construct bound to Kato III cells. The FACS staining was performed as described in Example 1A paragraph 5. The filled histogram represents cells incubated with the anti B7.1 antibody alone. The open histogram shows cells incubated with the construct and the anti B7.1 antibody.

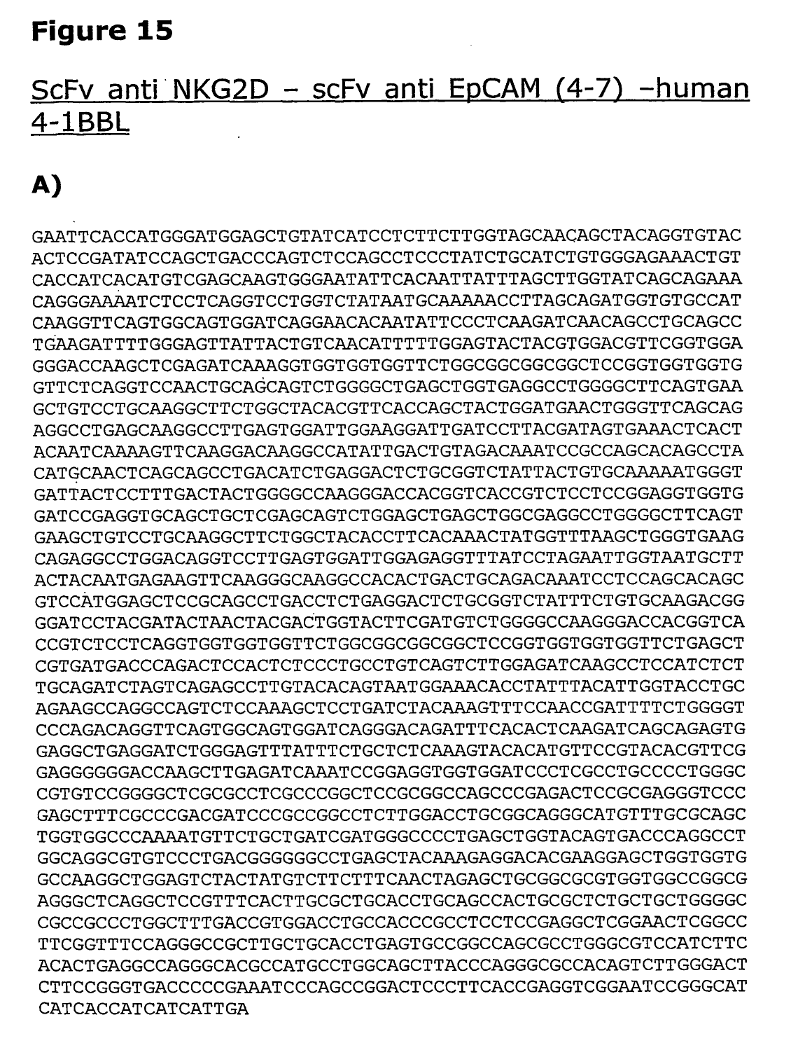

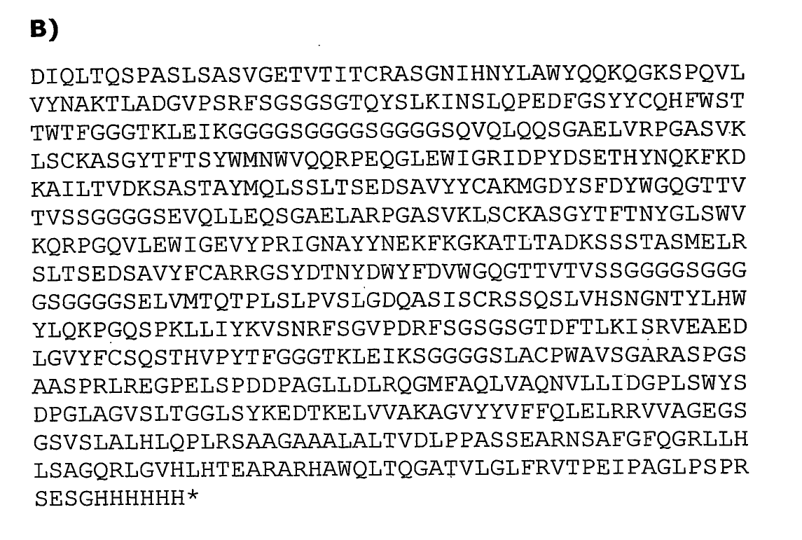

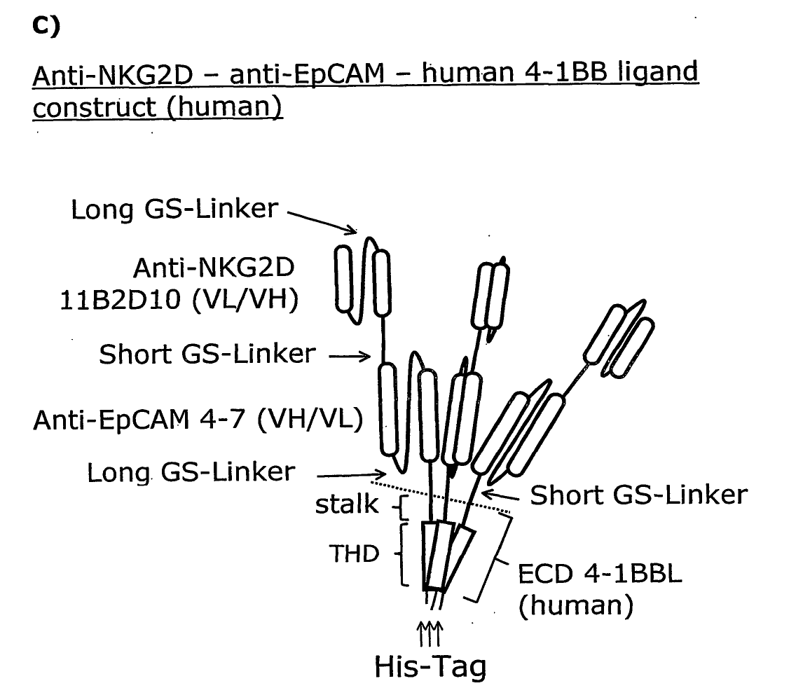

FIG. 15. Sequences of bispecific scFv (anti-NKG2Dxanti-EpCAM)×human 4-1BB ligand construct. A) Nucleotide sequence, B) protein sequence, C) schematic representation of the construct.

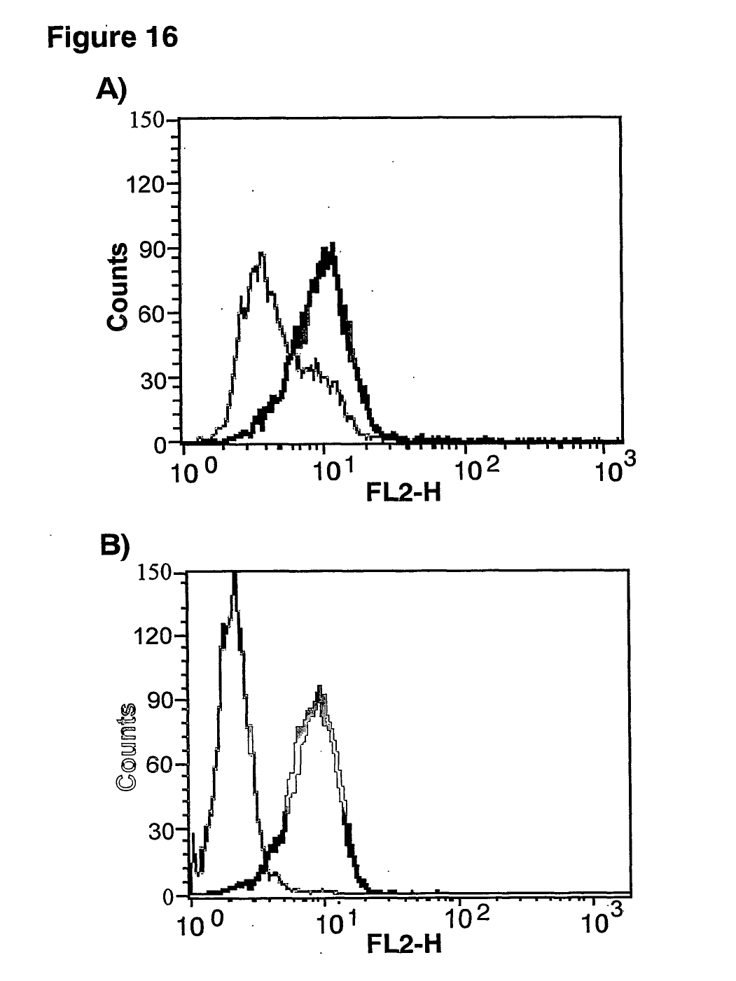

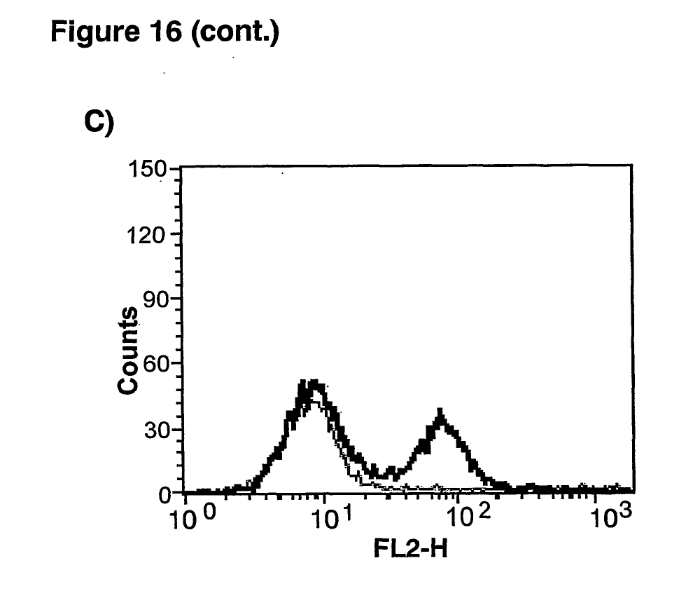

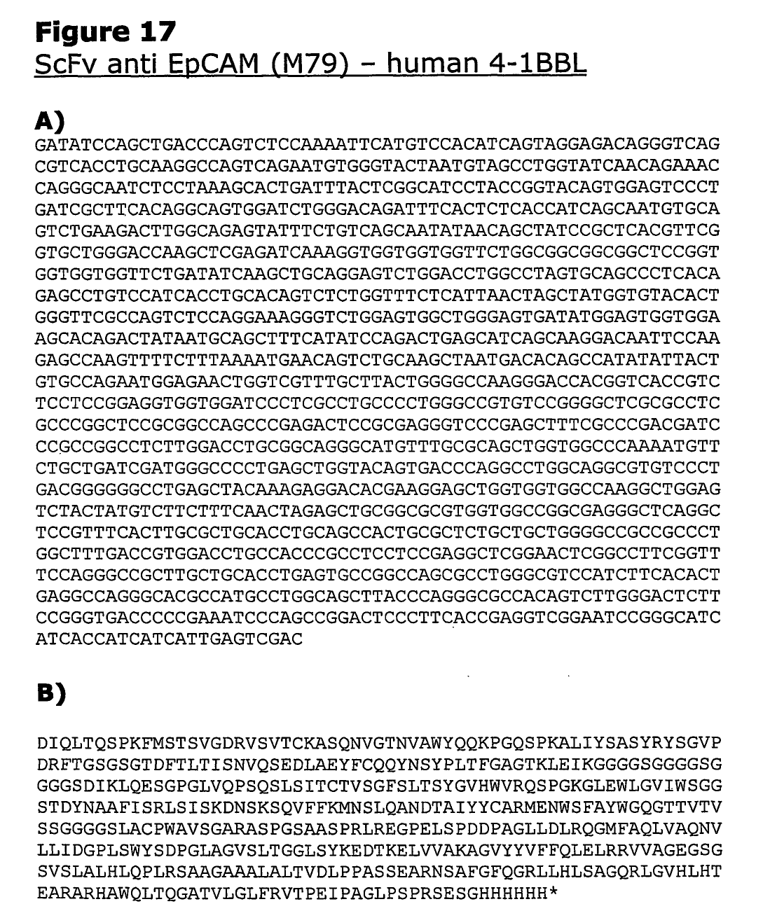

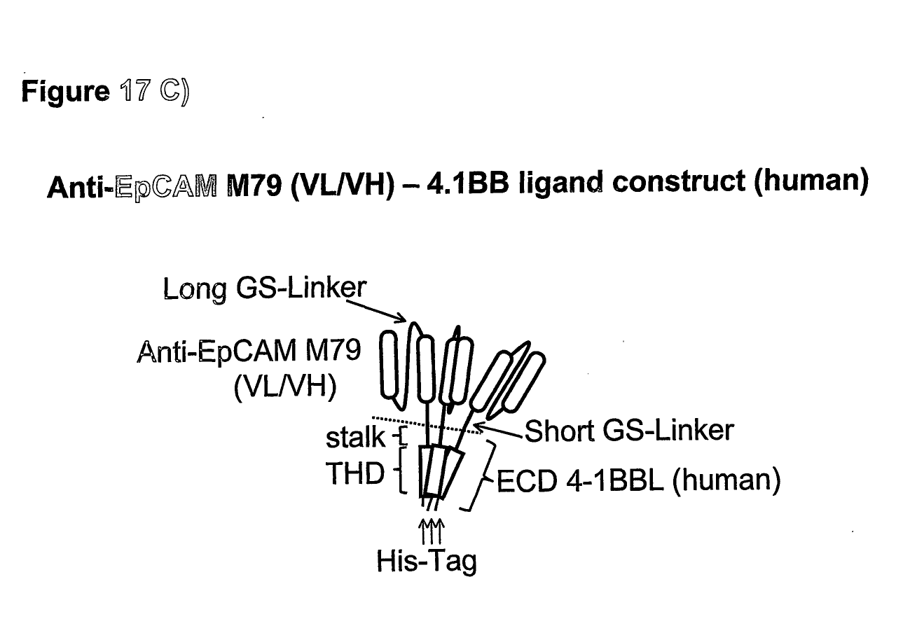

FIG. 16. Binding ability of the trifunctional construct anti-NKG2D—anti-EpCAM—human 4-1BB ligand on NKG2D+ CHO cells and EpCAM+ CHO cells (thick line) respectively. The detection of the bound construct was performed with the secondary antibody as stated below the histogram. As negative control untransfected CHO cells were used (thin line). Construct binding to NKG2D+ CHO cells. A) Detection via 4-1BBL antibody, B) detection via His-tag antibody. Construct binding to EpCAM+ CHO cells. C) Detection via 4-1BB ligand antibody. FIG. 17. Sequences scFv anti-EpCAM—human 4-1BB ligand construct. A) Nucleotide sequence, B) protein sequence, C) schematic representation of the construct.

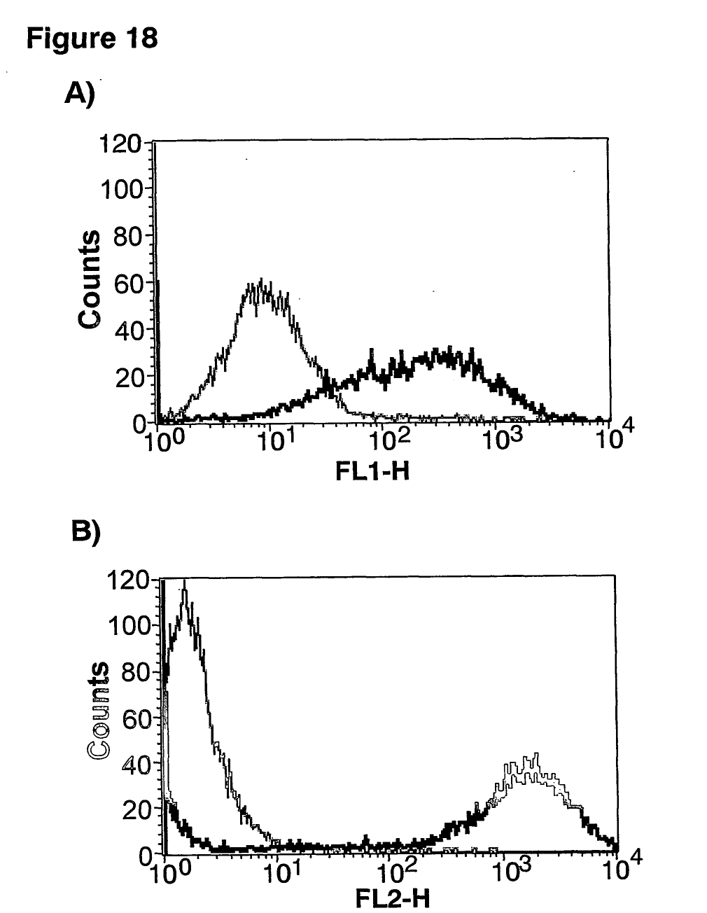

FIG. 18. Binding ability of the construct scFv anti-EpCAM—human 4-1BB ligand on EpCAM+ CHO cells (thick line). The detection of the bound construct was performed with the secondary antibody as stated below the histogram. As negative control the cell culture supernatant containing the secreted scFv anti-EpCAM—human 4-1BB ligand construct was not applied (thin line). A) Detection with anti-His Tag antibody. B) Detection with anti-4-1BB ligand antibody.

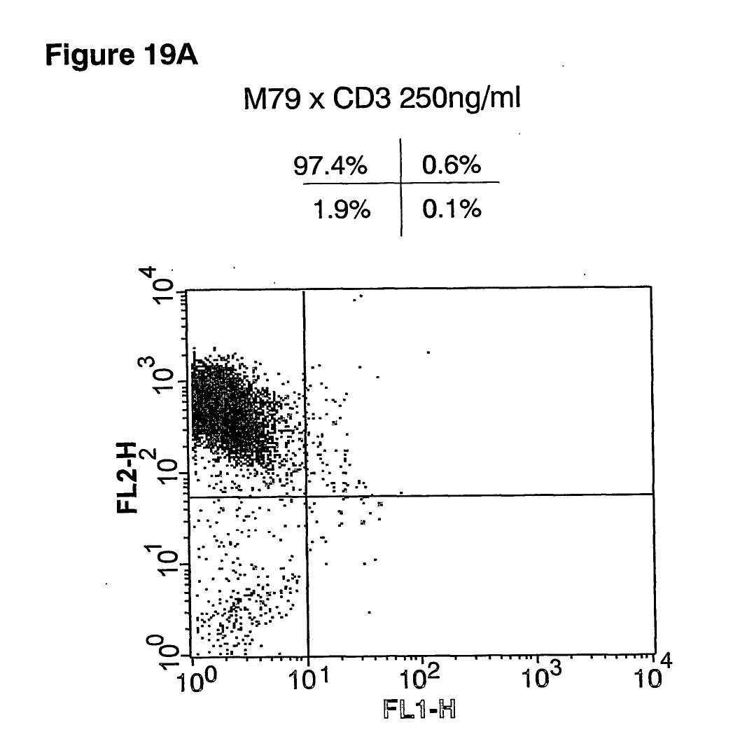

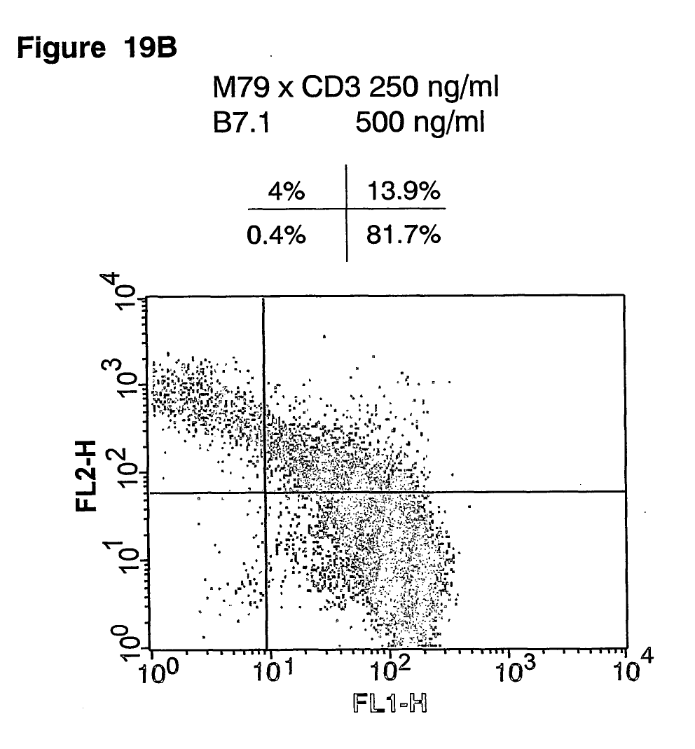

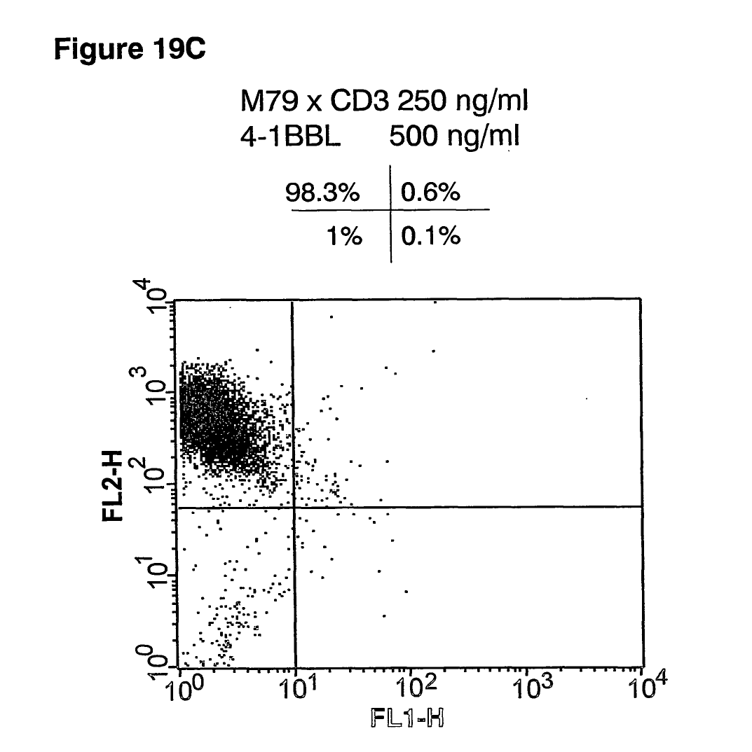

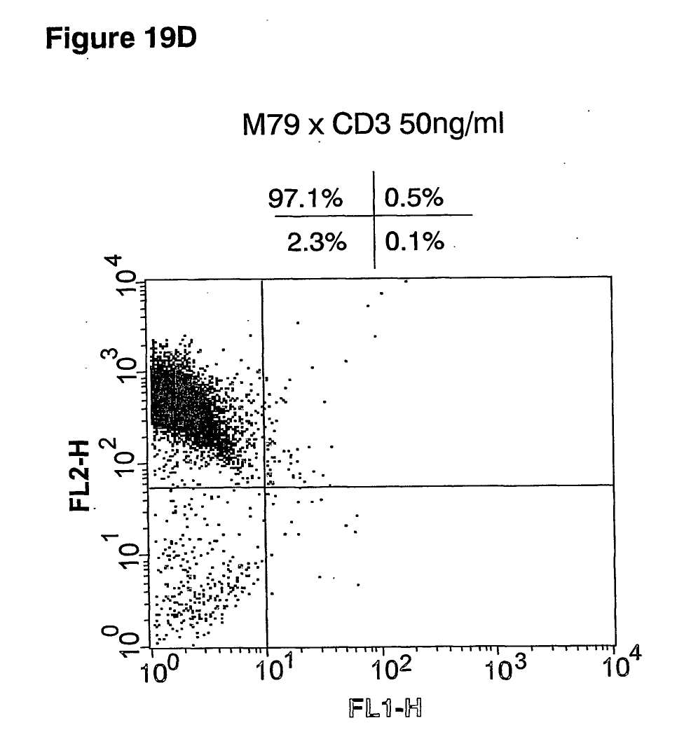

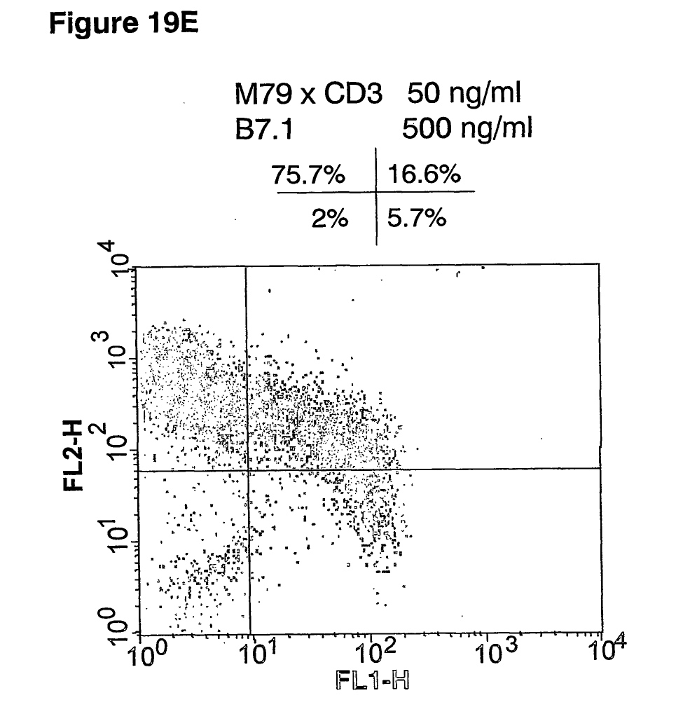

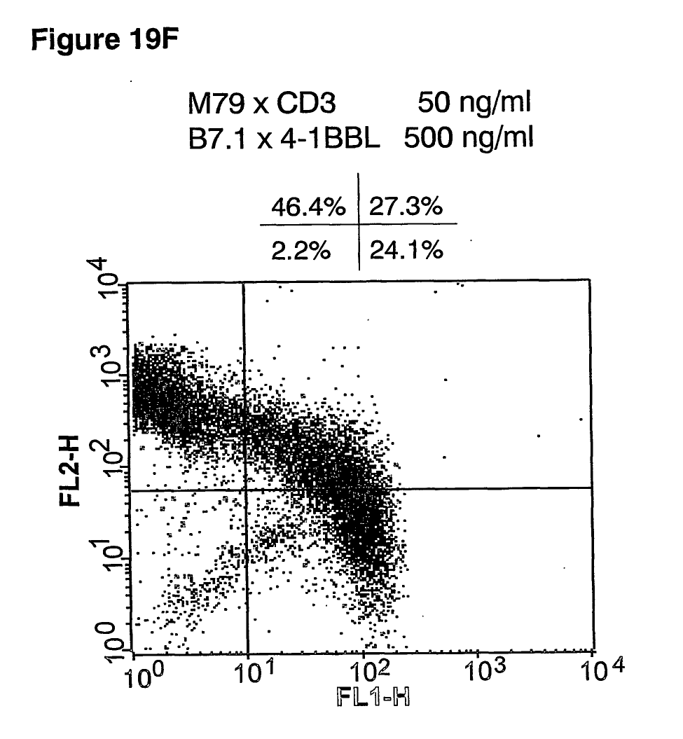

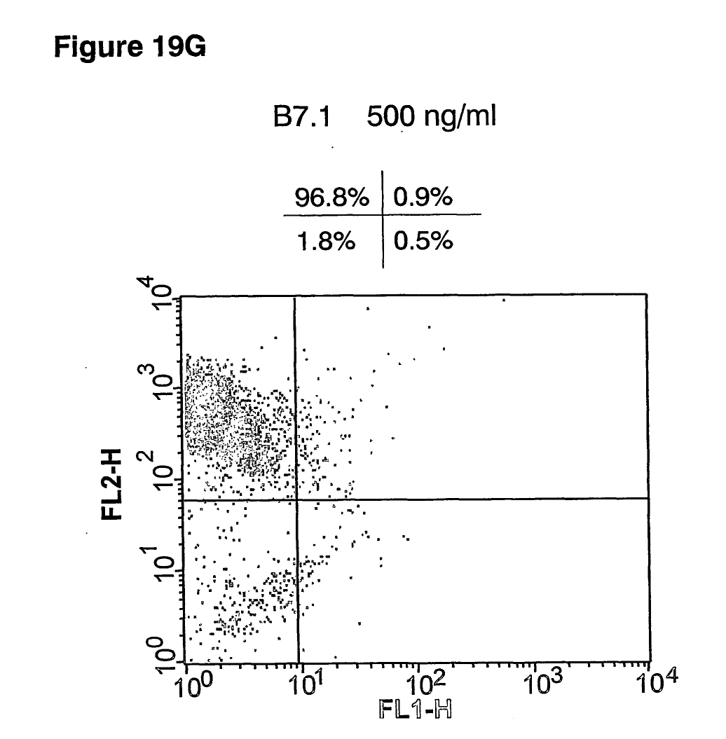

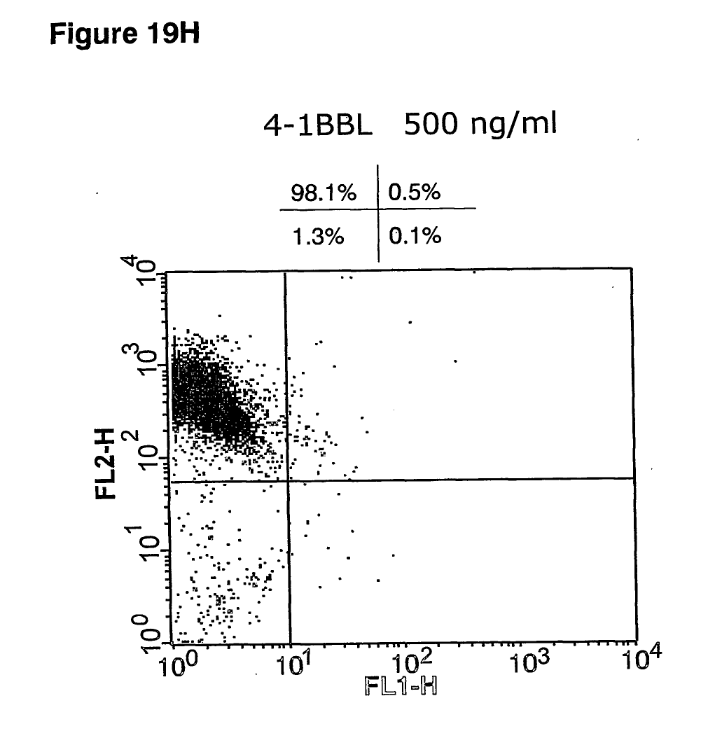

FIG. 19. FACS analysis/T cell priming A-l. All experimental data were taken after 6 days of culture. A) 1. signal: scFv antiEpCAM (M79)×scFv anti-CD3, 250 ng/ml. B) 1.+2. signal: scFv antiEpCAM (M79)×scFv anti-CD3, 250 ng/ml and B.7 −scFv antiEpCAM (4-7), 500 ng/ml. C) 1.+3. signal: scFv antiEpCAM (M79)×scFv anti-CD3, 250 ng/ml and scFv antiEpCAM (4-7)×hu4-1BBL, 500 ng/ml. D) 1. signal: scFv antiEpCAM (M79)×scFv anti-CD3, 50 ng/ml. E) 1.+2. signal: scFv antiEpCAM (M79)×scFv anti-CD3, 50 ng/ml and B.7 −scFv antiEpCAM (4-7), 500 ng/ml. F) 1.+2.+3. signal: scFv antiEpCAM (M79)×scFv anti-CD3, 50 ng/ml and B.7 −scFv antiEpCAM (4-7) —hu4-1BBL, 500 ng/ml. G) 2. signal: B.7 −scFv antiEpCAM (4-7), 500 ng/ml. H) 3. signal: B7.1 −scFv antiEpCAM (4-7), 500 ng/ml. l) 2.+3. signal: B.7 −scFv antiEpCAM (4-7) —hu4-1BBL, 500 ng/ml.

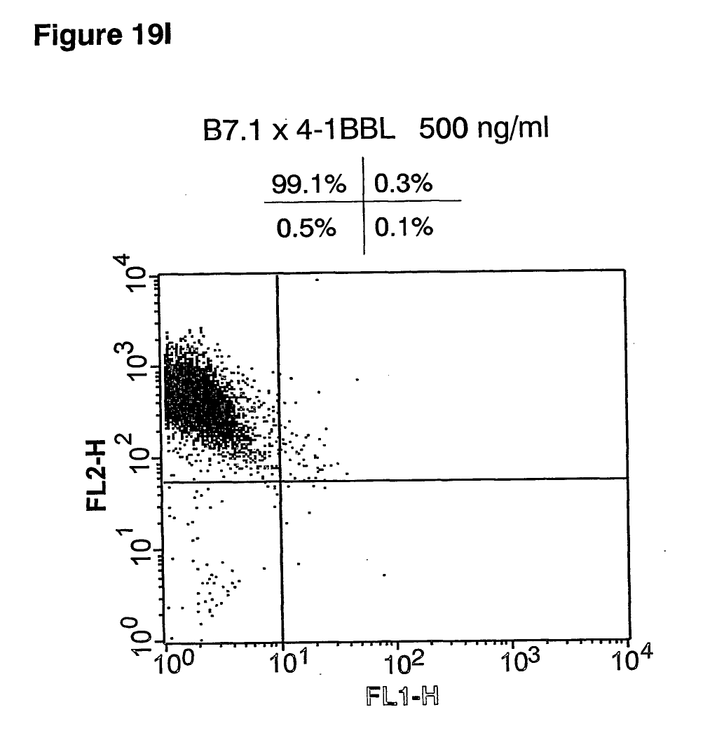

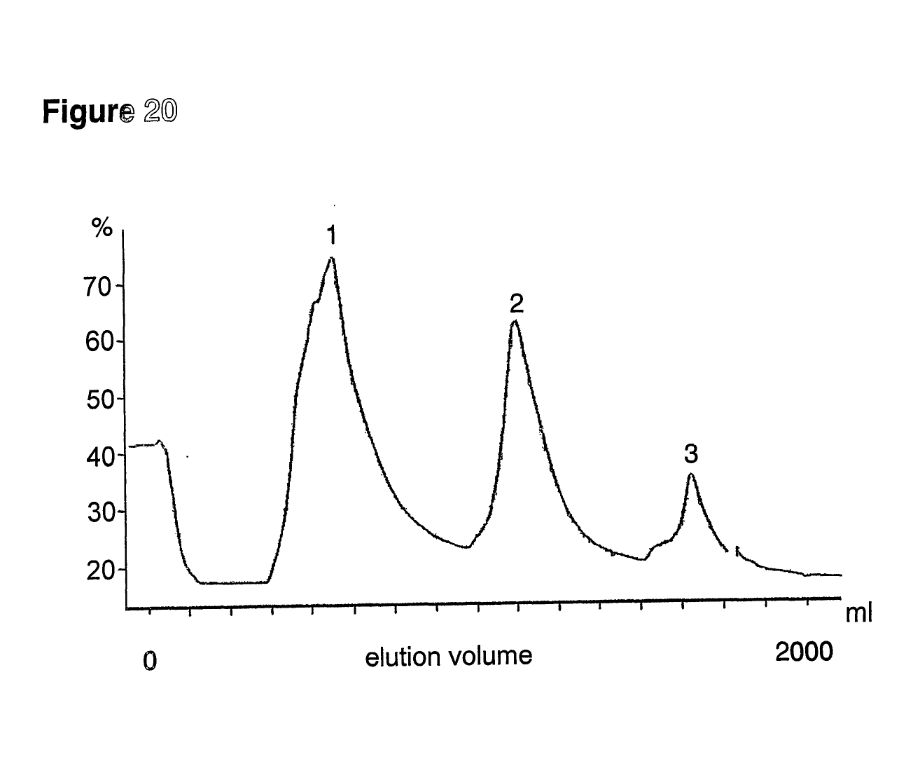

FIG. 20. Elution pattern of scFv anti-EpCAM (M79)—human 4-1.BB ligand fusion protein from an SP Sepharose cation exchange column. 1: protein eluted at 30% elution buffer B1; 2: protein eluted at 30% elution buffer B1; 3: protein eluted at 30% elution buffer B1. The protein peak from elution with 50% buffer B1 was used for further purification.

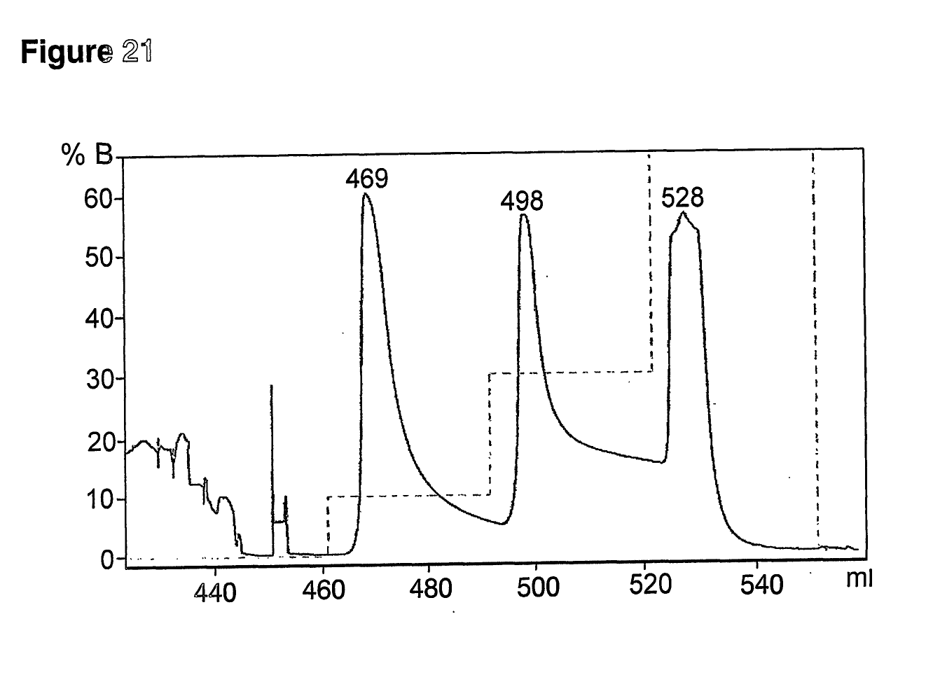

FIG. 21. Protein elution pattern from a Ni-chelating His trap column (bold line). ScFv anti-EpCAM (M79)—human 4-1.BB ligand fusion protein as contained in protein fractions of the protein peak from elution with 50% buffer B1 of previous SP sepharose cation exchange column was loaded. The broken line indicates the theoretical gradient of elution buffer containing 0.5 M imidazol. Protein fractions from the 30% buffer B2 elution step were used for further purification.

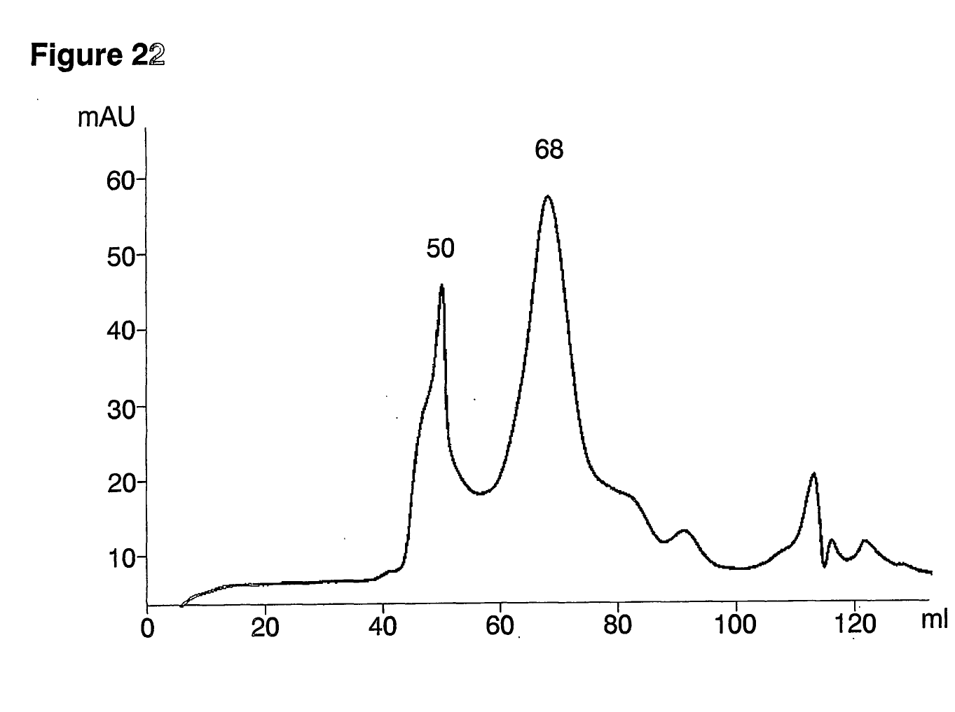

FIG. 22. Protein elution pattern (bold line) from a Sephadex S200 gelfiltration column. The scFv anti-EpCAM (M79)—human 4-1.BB ligand fusion protein elutes in a single peak at approx. 68 ml and corresponds to a molecular weight of approx.150 kDa.

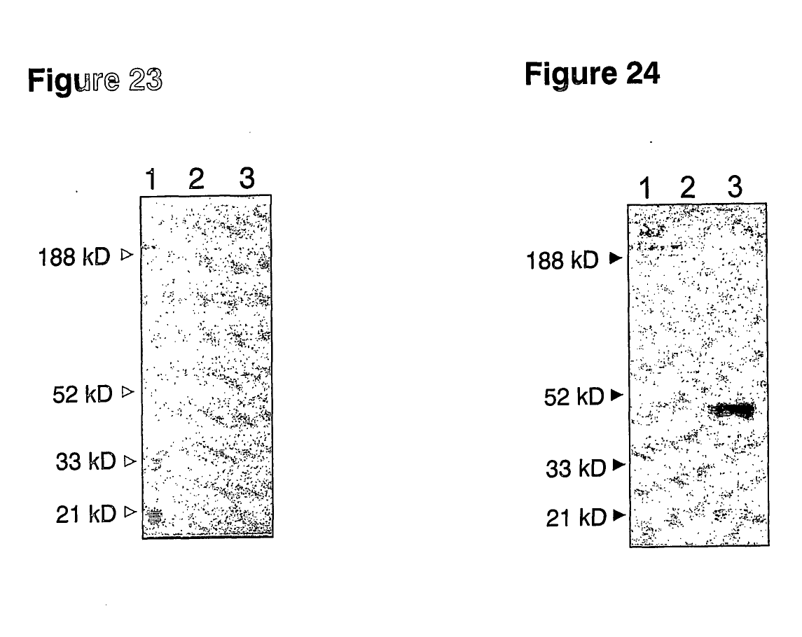

FIG. 23. SDS-PAGE analysis of purified scFv anti-EpCAM (M79)—human 4-1.BB ligand containing protein fractions as shown in FIG. 22. SDS-PAGE was stained with colloidal Coomassie. Lane 1: MultiMark molecular weight marker; lane 2 and 3: gelfiltration fractions of the main peak and the shoulder.

FIG. 24. Western blot analysis of purified scFv anti-EpCAM (M79)—human 4-1.BB ligand fusion protein fractions. The Western blot was incubated with Penta His antibody and goat anti-mouse antibody labeled with alkaline phosphatase. The stainer was BCIP/NBT liquid. Lane 1: MultiMark molecular weight marker; lane 2 and 3: gelfiltration fractions of the main peak at 68 ml in different concentrations. The main band at approx. 50 kDa contains>90% of the purified protein. The minor band at 21 kDa is a proteolytically cleaved fragment.

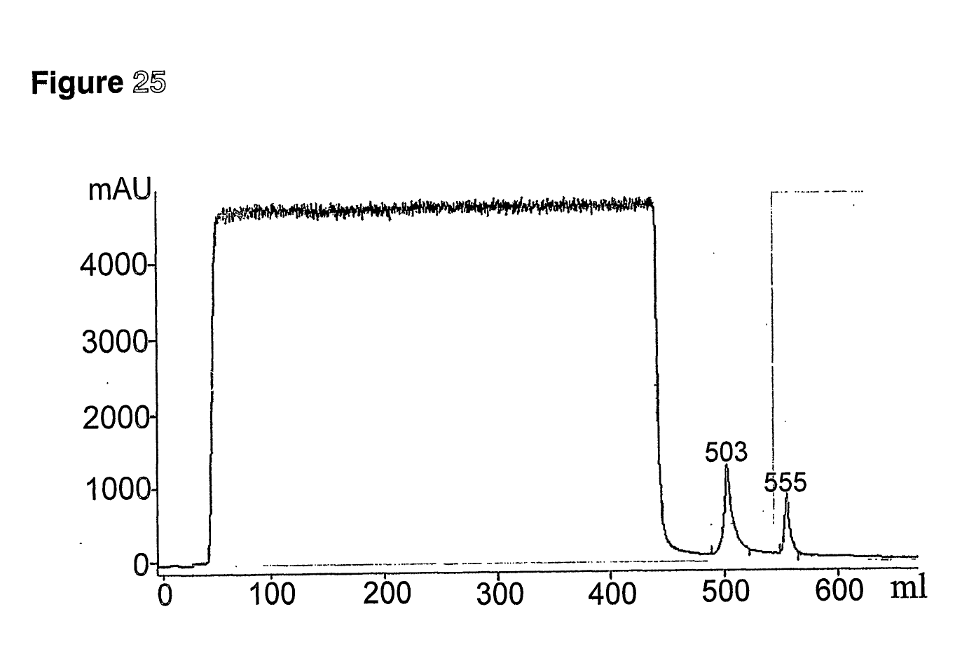

FIG. 25. Elution pattern of scFv anti-NKG2D—scFv anti-EpCAM (4.7)—human 4-1.BBL fusion protein containing protein fractions from a Ni-Chelating His Trap column (bold line). The grey line indicates the theoretical gradient of elution buffer. Protein fractions from the 100% buffer B2 elution step (peak at 555 ml) were used for further purification.

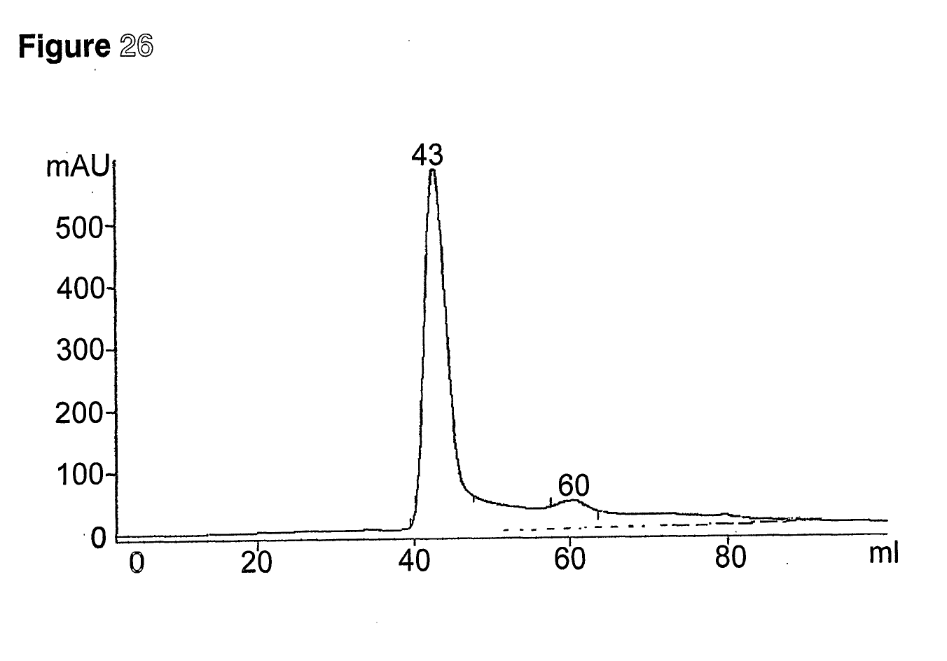

FIG. 26. Protein elution pattern (bold line) from a Sephadex S200 gelfiltration column. The scFv anti-NKG2D—scFv anti-EpCAM (4.7)—human 41BBL fusion protein elutes in a single peak at approx. 60 ml and corresponds to a molecular weight of approx. 220 kDa. The dotted line indicates the baseline.

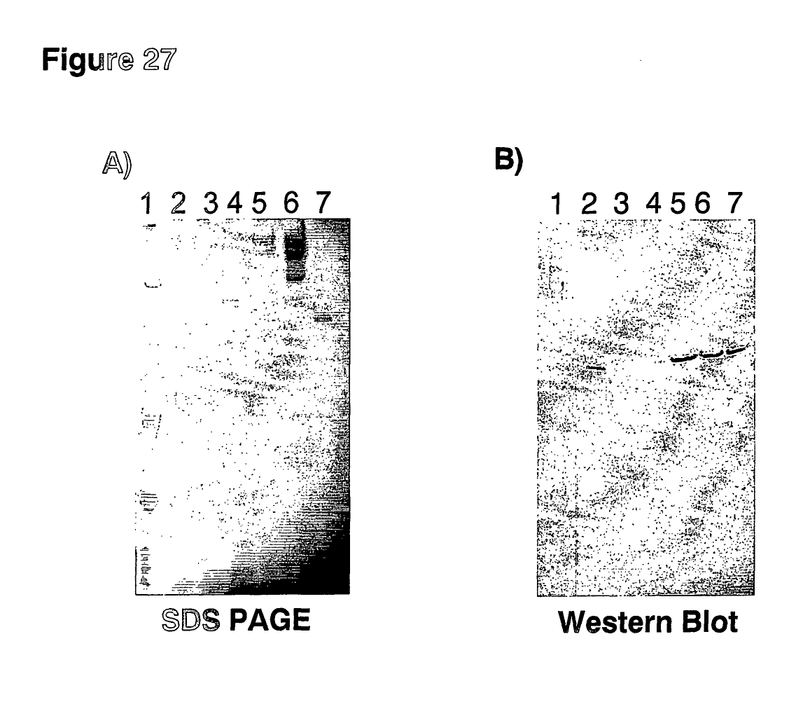

FIG. 27. SDS-PAGE (A) and Western blot (B) analysis of purified scFv anti-NKG2D—scFv anti-EpCAM (4.7)—human 41BBL fusion protein containing protein fractions. SDS-PAGE was stained with colloidal Coomassie. The Western blot was incubated with Penta His antibody and goat anti-mouse antibody labeled with alkaline phosphatase. The stainer was BCIP/NBT liquid. Lane 1: MultiMark molecular weight marker, lane 2: cell culture supernatant, lane 3: IMAC flow through, lane 4: IMAC wash peak, lane 5: IMAC eluate peak, lane 7: gelfiltration fractions of the peak at 60 ml. The main band at approx. 72 kDa contains the protein in>50% purity.

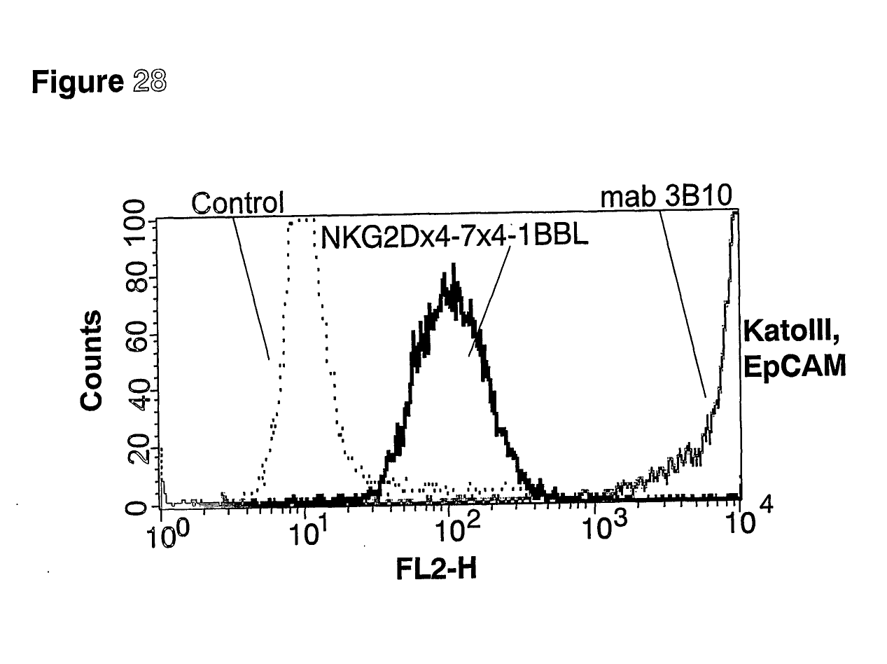

FIG. 28. FACS binding analysis of the scFv anti-NKG2D—scFv anti-EpCAM (4.7)—human 41BBL fusion protein to the EpCAM antigen on Kato III cells. The FACS staining was performed as described in Example 1A paragraph 4. The dotted line represents a control, whereby cells were incubated with the anti-his antibody and the second step reagent alone. The bold line shows cells incubated with the scFv anti-NKG2D—scFv anti-EpCAM (4.7)—human 41BBL fusion protein from cell culture supernatant. The thin line represents a positive control, whereby cells were incubated with the anti-EpCAM antibody mab 3B10.

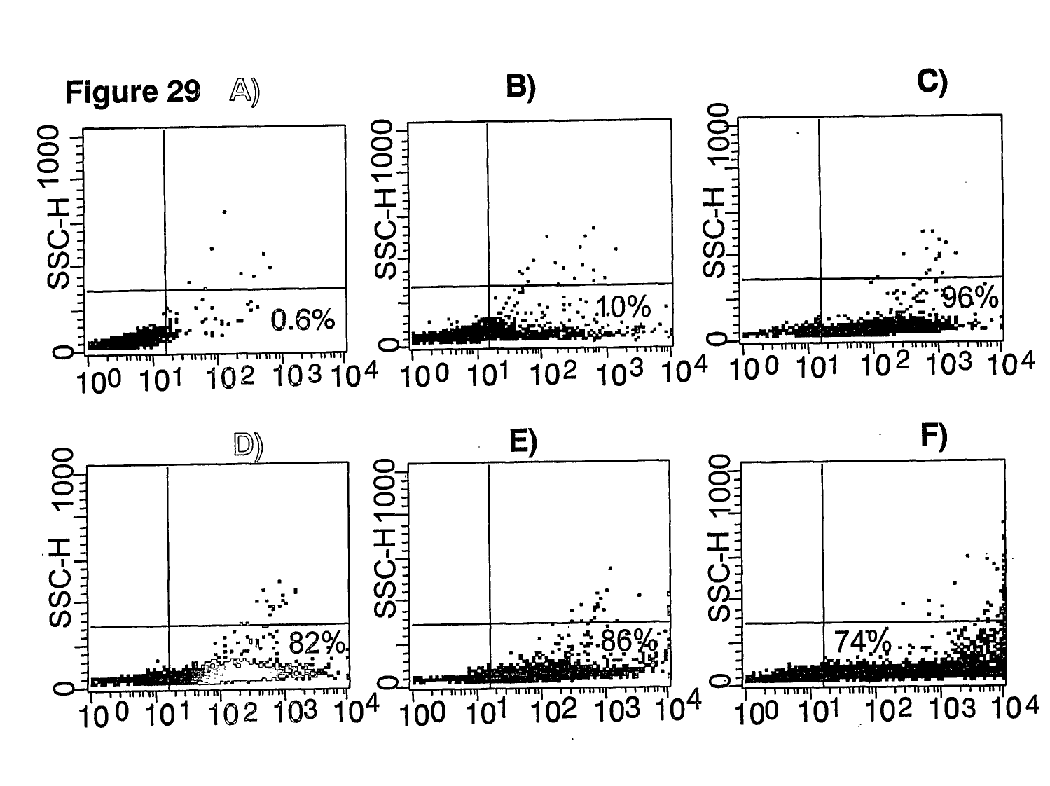

FIG. 29. NKG2D binding assay: (A) NK control unstained; (B) NK control detection antibodies; (C) NK NKG2D (1D11) mab; (D) NK NKG2D (11B2D10) mab; (E) NK cell culture supernatant; (F) NK CD16 mab. The x-achses is in all cases fluorescence 2 (FL2-H). The y-achses is in all cases sideward scatter (SSC-H). The invention will now be described by reference to the following biological examples which are merely illustrative and are not to be construed as a limitation of scope of the present invention.

### EXAMPLE 1

Generation of an scFv Anti-237—Murine 4-1BB Ligand Construct

The cDNA of murine 4-1BB ligand was isolated from murine splenocytes. The isolation of total RNA and cDNA synthesis by random-primed reverse transcription was performed according to standard protocols (Sambrock, Molecular Cloning; A Laboratory Manual, 2nd edition, Cold Spring Harbour laboratory Press, cold Spring Harbour, N.Y. (1989)). A PCR (denaturation at 93° C. for 5 min, annealing at 58° C. for 1 min, elongation at 72° C. for 1 min for the first cycle; denaturation at 93° C. for 1 min, annealing at 58° C. for 1 min, elongation at 72° C. for 1 min for 30 cycles; termial extension at 72° C. for 5 min) was used to amplify the coding sequence of the extracellular domain of murine 4-1BB ligand. The primers (5′ murine 4-1BB ligand: CGGGATCCCGCACCGAGCCTCGG (SEQID 1); 3′ murine 4-1BB ligand: GGATCCGGATTCCCATGGGTTGTCGGGTTTC (SEQID 2) used in the PCR were designed as to introduce restriction sites at the beginning and the end of the cDNA coding for the extracellular portion of murine 4-1BB ligand (SEQID 3 and 4). The introduced restriction sites, BamHl and BspEl, were utilised in the following cloning procedures. The amplified cDNA coding for the extracellular portion of murine 4-1BB ligand was then cloned via BamHl and BspEl into a plasmid designated as BSCTI to attach a sequence to the c-terminus coding for a polyhistidine tag of six consecutive histidine residues followed by a stop codon (BSCTI is described in Kufer et al. Cancer immunity Vol. 1, p. 10 (12 November 2001)). In this step the BspEl site of the cDNA was fused into a Xmal site of the plasmid thereby destroying both sites. By cloning into BSCTI there was also attached a sequence coding for a glycine-serine linker [(Ser-Gly4-Ser)1] to the N-terminus of the 4-1BB ligand sequence. The sequence of different clones was determined by sequencing according to standard protocols (Sambrock, Molecular Cloning; A Laboratory Manual, 2nd edition, cold Spring Harbour laboratory Press, Cold Spring Harbour, N.Y. (1989)). The modified and verified cDNA sequence was then cloned into a Plasmid designated pEFDHFR (pEFDHFR was described in Mack et al. Proc. Natl. Acad. Sci USA 92 (1995) 7021-7025). Differing from the original pEFDHFR this plasmid already contained the cDNA sequence coding for the 237 single chain antibody (parental anti-237 antibody published as PW237 in “Ward et al., 1989, J. Exp. Med. Volume 170, 217-232; SEQID 5 & 6) binding to a tumour specific cell surface antigen on the murine sarcoma cell line designated AG104A (the cell line was described in Wick et al. J. Exp. Med. Volume 186, Number 2, July 21, 1997 229-238). The 237 cDNA sequence was positioned in the plasmid as to allow for secreted expression in eukaryotic cells. For the cloning of the cDNA of murine 4-1BB ligand into pEFDHFR the restriction enzymes BspEl and Sall were used. In the modified 4-1BB ligand sequence the recognition sequence of BspEl is positioned at the beginning of the afore mentioned glycine-serine linker whereas the recognition site for Sall is postioned after the stop codon following the polyhistidine tag. By the described cloning step the cDNA of the extracellular portion of murine 4-1BB ligand was fused to the 3′ end of the cDNA of the 237 single chain antibody. The plasmid contained now a bifunctional construct comprised of the cDNA sequence coding for the anti-237 single chain antibody followed by the sequence coding for the extracellular portion of murine 4-1BB ligand (FIG. 3). SEQ ID NO: 7 and 8 show the sequence of the construct without His-tag. All cloning steps were designed as to generate an intact reading frame for the bifunctional construct.

Expression of the scFv Anti-237—Murine 4-1BB Ligand Construct

The plasmid with the sequence coding for the bifunctional construct was transfected into DHFR deficient CHO cells for eukaryotic expression of the construct (pEFDHFR was described in Mack et al. Proc. Natl. Acad. Sci. USA 92 (1995) 7021-7025 and eukaryotic protein expression in DHFR deficient CHO cells was performed as described in Kaufmann R. J. (1990) Methods Enzymol. 185, 537-566). Geneamplification of the construct was induced by increasing concentrations of MTX up to a final concentration of 500 nM MTX. The transfected cells were then expanded and supernatant produced for purification.

Purification of the scFv Anti-237—Murine 4-1BB Ligand Construct

The anti-237scFv—4-1BB ligand construct protein was isolated from cell culture supernatant in a three step purification process including cation exchange chromatography (FIG. 4), immobilized metal affinity chromatography (IMAC) (FIG. 5) and gelfiltration (FIG. 6). Äkta FPLC System and GradiFrac (Pharmacia, Tennenlohe, Germany) and Unicorn Software were used for chromatography. All chemicals were of research grade and purchased from Sigma (Deisenhofen, Germany) or Merck (Darmstadt, Germany).

Cation exchange was performed on an SP Sepharose column (Pharmacia, Tennenlohe, Germany) that was equilibrated with buffer A1 (20 mM MES pH 5.5). Cell culture supernatant was diluted 1:3 with buffer A1 and applied to the column (bedsize 300 ml, packed in a XK column, Pharmacia, according to the manufacturers protocol) with a flow rate of 20 ml/min at 4° C. Unbound sample was washed out with buffer A1 and the bound protein was eluted with a three step gradient of 25%, 50% and 100% buffer B1 (20 mM MES pH 5.5, 1M NaCl) in volumes of two CV. Eluted protein fractions from the 50% B1 step were pooled for further purification (FIG. 4).

IMAC was performed, using a HisTrap 5 ml column (Pharmacia, Tennenlohe, Germany) that was preloaded with NiSO4 according to the manufacturers protocol. The column was equilibrated with buffer A2 (20 mM NaPP pH 7.2, 0.4 M NaCl). The sample was applied to the column with a flow rate of 1 ml/min and the column was washed with buffer A2 to remove unbound sample. Bound protein was eluted using a three step gradient of buffer B2 (20 mM NaPP pH 7.0, 0.4 M NaCl, 0.5 M Imidazol) Step 1: 10% buffer B2, step 2: 30% buffer B2, step 3: 100% buffer B2, each step for 4 column volumes. Eluted protein fractions from the third step were pooled for futher purification (FIG. 5).

Gelfiltration chromatography was performed on a Sephadex S200 HiPrep column (Pharmacia, Tennenlohe, Germany) equilibrated with PBS (Gibco Invitrogen Corp., Carlsbad, USA) (FIG. 6). The column was previously calibrated for molecular weight determination (molecular weight marker kit MW GF-200, Sigma-Aldrich Chemie GmbH, Munich, Germany). Eluted protein samples (flow rate 1 ml/min) were subjected to SDS-Page and Western Blot for detection. Protein concentrations were determined using absorption values at 280 nm in combination with the molar absorption coefficient. The final product has an apparent molecular weight of ca. 50 kDa on SDS PAGE (FIG. 7) and Western Blot (FIG. 8).

SDS PAGE under reducing conditions was performed with precast 4-12% Bis Tris gels (Invitrogen GmbH, Karlsruhe, Germany). Sample preparation and application were according to the manufacturers protocol. The molecular weight was determined with MultiMark protein standard (Invitrogen GmbH, Karlsruhe, Germany). The gel was stained with colloidal Coomassie according to Invitrogen protocol.

Western Blot was performed with a BioTrace membrane (Pall Life Sciences) and the Invitrogen Blot Module according to the manufacturers protocol. The antibodies used were Penta His (Quiagen) and Goat-anti-Mouse-AP (Sigma), the stainer was BCIP/NBT liquid (Sigma).

In conclusion, the molecular weight of the peak observed with gelfiltration chromatography is 150 kDa. This corresponds to three times the molecular weight of the monomer, which is 50 kDa as observed with SDS PAGE (FIG. 7) and Western Blot (FIG. 8). That corresponds to a trimeric form of anti-237 scFv—4-1BB ligand construct. These results clearly demonstrate that the polypeptide construct of the present invention is a trimer.

The purity of the isolated protein was>95% as determined by SDS-PAGE (FIG. 7). The final yield of purified protein was ca. 5.5 mg/I cell culture supernatant.

FACS Assay for scFv Anti-237 Binding on Ag104A Cells

Binding of the purified bifunctional construct to the tumour specific cell surface antigen on the AG104A cell line was tested using an FACS assay. For that purpose a number of 2,5*10⁵ cells was incubated with 10 μg/ml of the construct in 50 μl PBS with 2% FCS. The binding of the construct was detected with an anti-His antibody (Penta-His Antibody, BSA free, obtained from Quiagen GmbH, Hilden, FRG) at 2 μg/ml in 50 μl PBS with 2% FCS. As a second step reagent a R-Phycoerythrin-conjugated affinity purified F(ab′)₂ fragment, goat anti-mouse IgG, Fc-gamma fragment specific antibody, diluted 1:100 in 50 μl PBS with 2% FCS (obtained from Dianova, Hamburg, FRG) was used. The samples were measured on a FACSscan (BD biosciences, Heidelberg, FRG). Antigen binding was clearly detectable (FIG. 9).

Detection of the 4-1BB Ligand Portion of the Construct

The presence of the 4-1BB ligand portion of the construct was demonstrated by a FACS based assay. For that purpose the AG104A cell line which shows no surface expression of murine 4-1BB ligand was used. A number of 2,5*105 cells was incubated with 10 μg/ml of the construct in 50 μl PBS with 2% FCS. The presence of the 4-1BB ligand portion was detected with an anti-murine 4-1BB ligand antibody (purified rat anti-mouse 4-1BB ligand monoclonal antibody obtained from BD biosciences, Heidelberg, FRG) at 5 μg/ml in 50 μl PBS with 2% FCS. As a second step reagent a R-Phycoerythrin-conjugated affinity purified F(ab′)2 fragment, goat anti-rat IgG, Fc-gamma fragment specific antibody, diluted 1:100 in 50 μl PBS with 2% FCS (obtained from Dianova, Hamburg, FRG) was used. The samples were measured on a FACSscan (BD biosciences, Heidelberg, FRG). Presence of the 4-1BB ligand antigen on the AG104A cells, resulting from the binding of the construct, was clearly detectable (FIG. 10).

### EXAMPLE 2

Cloning of Human 4-1BB Ligand

The cDNA of human 4-1BB ligand was isolated from human monocytes differentiated into dendritic cells by stimulation with GM-CSF and IL-4 (as described in de Baey et al. Eur J Immunol 2001 Jun; 31(6):1646-55). The isolation of total RNA and cDNA synthesis by random-primed reverse transcription was performed according to standard protocols (Sambrock, Molecular Cloning; A Harbour, New York (1989)). A PCR (denaturation at 96° C. for 5 min, annealing at 58° C. for 1 min, elongation at 72° C. for 1 min for the first cycle; denaturation at 96° C. for 1 min, annealing at 58° C. for 1 min, elongation at 72° C. for 1 min for 30 cycles; termial extension at 72° C. for 5 min) was used to amplify the coding sequence of the extracellular domain of human 4-1BB ligand. The primers (5′ human 4-1BB ligand: CGGGATCCCTCGCCTGCCCCTGGGCC (SEQID 9); 3′ human 4-1BB ligand: GGATCCGGATTCCGACCTCGGTGMGGGAG (SEQID 10)) used in the PCR were designed as to introduce restriction sites at the beginning and the end of the cDNA coding for the extracellular portion of human 4-1BB ligand (SEQID 11 and 12). The introduced restriction sites, BamHl and BspEl, were utilised in the following cloning procedures. The amplified cDNA coding for the extracellular portion of human 4-1BB ligand was then cloned via BamHl and BspEl into a plasmid designated as BSCTI to attach a sequence to the c-terminus coding for a polyhistidine tag of six consecutive histidine residues followed by a stop codon (BSCTI is described in Kufer et al. Cancer immunity Vol. 1, p. 10 (12 Nov. 2001)). In this step the BspEl site of the cDNA was fused into a Xmal site of the plasmid thereby destroying both sites. By cloning into BSCTI there was also attached a sequence coding for a glycine-serine linker [(Ser-Gly4-Ser)1] to the n-terminus of the 4-1BB ligand sequence (Linker sequence). The sequence of different clones was determined by sequencing according to standard protocols (Sambrock, Molecular Cloning; A Laboratory Manual, 2nd edition, Cold Spring Harbour laboratory Press, cold Spring Harbour, N.Y. (1989)).

Generation of a B7.1-anti-EpCAM scFv (4-7)—human 4-1BB Ligand Construct

The modified and verified cDNA sequence encoding human 4-1BB ligand was then cloned into a plasmid designated B7.1/4-7 pEFDHFR (described in Kufer et al. Cancer immunity Vol. 1, p. 10 (12 Nov. 2001)) replacing the 4-7 fragment. For this purpose the restriction enzymes BspEl and Sall were used. In the modified 4-1BB ligand sequence the recognition sequence of BspEl is positioned at the beginning of the afore mentioned glycine-serine linker whereas the recognition site for Sall is postioned after the stop codon following the polyhistidine tag. The plasmid B7.1/4-7 pEFDHFR contains a cDNA sequence coding for the extracellular portion of the human B7.1 molecule. This sequence was positioned in the plasmid as to allow for expression in eukaryotic cells. By the described cloning step the cDNA of the extracellular portion of human 4-1BB ligand was fused to the cDNA of B7.1. Into the BspEl site, now positioned between the B7.1 and the 4-1BB ligand sequence, another sequence coding for the 4-7 single chain antibody binding to the extracellular portion of the EpCAM Antigen was inserted. For this purpose, the sequence coding for the 4-7 single chain antibody was modified using a PCR (denaturation at 93° C. for 5 min, annealing at 58° C. for 1 min, elongation at 72° C. for 1 min for the first cycle; denaturation at 93° C. for 1 min, annealing at 58° C. for 1 min, elongation at 72° C. for 1 min for 30 cycles; termial extension at 72° C. for 5 min). The primer set (5′ scFv4-7: CATTTTCCTGATMCTCCGGAGGTGG (SEQID 13); 3′ scFv4-7: AATCCGGATTTGATCTCAAGCTTGGTCCC (SEQID 14)) generated for this PCR was designed as to create two flanking BspEl sites. In the PCR the sequence coding for an N-terminal glycine-serine linker [(Ser-Gly4-Ser)1] attached to the 4-7 single chain antibody present in the template was retained. The amplified sequence was then cloned into the afore mentioned BspEl site. The orientation and sequence of the insert was verified by sequencing according to standard protocols (Sambrock, Molecular Cloning; A Laboratory Manual, 2nd edition, Cold Spring Harbour laboratory Press, cold Spring Harbour, N.Y. (1989)). The plasmid contained now a trifunctional construct comprising the extracellular portion of human B7.1 fused to the sequence coding for the 4-7 single chain antibody followed by the sequence coding for the extracellular portion of human 4-1BB ligand. All cloning steps were designed as to generate an intact reading frame for the trifunctional construct (FIG. 11). SEQ ID NO: 15 and 16 show the sequence of the construct without His-tag.

Expression of the B7.1—anti-EpCAM scFv (4-7)—Human 4-1BB Ligand Construct in CHO Cells

The plasmid with the sequence coding for the trifunctional construct was transfected into DHFR deficient CHO cells for eukaryotic expression of the construct (pEFDHFR was described in Mack et al. Proc. Natl. Acad. Sci. USA 92 (1995) 7021-7025 and eukaryotic protein expression in DHFR deficient CHO cells was performed as described in Kaufmann R. J. (1990) Methods Enzymol. 185, 537-566). Geneamplification of the construct was induced by increasing concentrations of MTX up to a final concentration of 100 nM MTX. The transfected cells were then expanded and 10 litres of supernatant produced. The construct was finally purified out of the culture supernatant (purification was performed as described in Kufer et al. Cancer immunity Vol. 1, p. 10 (12 Nov. 2001))

Assay for EpCAM Binding

Binding of the purified trifunctional construct to the extracellular portion of the EpCAM antigen was tested using an FACS assay. For that purpose the EpCAM positive human gastric cancer cell line Kato III (obtained from American Type Culture Collection (ATCC) Manassas, Va. 20108 USA, ATCC number: HTB-103) was used. Cells were cultured according to the recommendations of the supplier and a number of 2,5*10⁵ cells was incubated with 10 μg/ml of the construct in 50 μl PBS with 2%FCS. The binding of the construct was detected with an anti-His antibody (Penta-His Antibody, BSA free, obtained from Quiagen GmbH, Hilden, FRG) at 2 μg/ml in 50 μl PBS with 2% FCS. As a second step reagent a R-Phycoerythrin-conjugated affinity purified F(ab′)₂ fragment, goat anti-mouse IgG, Fc-gamma fragment specific antibody, diluted 1:100 in 50 μl PBS with 2% FCS (obtained from Dianova, Hamburg, FRG) was used. The samples were measured on a FACSscan (BD biosciences, Heidelberg, FRG). EpCAM binding was clearly detectable (FIG. 12).

Detection of the 4-1BB Ligand Portion and the B7.1 Portion of the Construct

The presence of the 4-1BB ligand portion and the B7.1 portion of the construct was demonstrated by a FACS based assay. For that purpose the EpCAM positive human gastric cancer cell line Kato III (obtained from ATCC, see above), which shows no surface expression of human B7.1 and human 4-1BB ligand was used. A number of 2,5*10⁵ cells was incubated with 10 μg/ml of the construct in 50 μL PBS with 2% FCS. The presence of the 4-1BB ligand portion was detected with an R-Phycoerythrin-conjugated mouse anti human 4-1BB ligand antibody (BD biosciences, Heidelberg, FRG), diluted 1:10 in 50 μl PBS with 2% FCS. The samples were measured on a FACSscan (BD biosciences, Heidelberg, FRG). Presence of the 4-1BB ligand antigen on the Kato III cells, resulting from the binding of the construct, was clearly detectable (FIG. 13). For the detection of the B7.1 portion an assay with the same conditions except for the use of a Phycoerythrin-conjugated mouse anti human B7.1 antibody (BD biosciences, Heidelberg, FRG), diluted 1:10 in 50μl PBS with 2% FCS, was performed. Presence of the B7.1 antigen on the Kato III cells, resulting from the binding of the construct, was clearly detectable (FIG. 14).

### EXAMPLE 3

Generation of a bispecific scFv 4-1BB Ligand Construct: anti-NKG2D—anti-EpCAM—human 4-1BB Ligand

The existing construct as described in example 2 was the basis for generation of a second trispecific construct: Anti-NKG2D—anti-EpCAM—4-1BB ligand, which is depicted schematically in FIG. 15C. This construct has a different mode of action: It redirects the cytotoxic activity of NKG2D positive CTLs and NK-cells to EpCAM positive cancer cells.

The isolation of the binding site specifically recognizing an extracellular epitope of the NKG2D receptor complex was described in the patent application WO 0171005 (Multifunctional Polypeptides Comprising A Binding Site To An Epitope Of The NKG2D Receptor Complex). As described in detail in example 3 of WO0171005 the NKG2D binding site is flanked by the restriction enzymes BsrGl/BspEl which were used to clone the NKG2D scFv-fragment into the mammalian expression vector pEF-DHFR already containing the coding sequence for the anti-EpCAM specificity 4-7 and the 4.1BB ligand. The resulting antibody construct (SEQID 17 and 18) with the domain arrangement VL_{anti-NKG2D (11B2D10)—}VH_{anti-NKG2D (11B2D10)}—VH_{anti-EpCAM (4-7)}—VL_{anti-EpCAM (4-7)}—Extracellular Domain_{4.1BB ligand} was transfected and expressed in CHO cells according to the example 2. Purification was performed as described (Kufer et al., 2001, Cancer Immunity, 10). The sequence and a schematic representation of this construct are shown in FIG. 15. SEQ ID NO: 17 and 18 show the sequence of the construct without His-tag.

Flowcytometric Binding Analysis of anti-NKG2D—anti-EpCAM—Human 4-1BB Ligand Construct

In order to test the functionality of the construct with regard to binding capability a FACS analysis was performed. For this purpose CHO transfectants were generated which were expressing the extracellular domains of the NKG2D and EpCAM antigen, respectively. 200,000 NKG2D+CHO cells and 200,000 EpCAM+CHO cells respectively were incubated with 50 μl the pure cell culture supernatant of CHO cells transfected with the Anti-NKG2D—anti-EpCAM—4.1 BB ligand construct for 30 min on ice. The cells were washed subsequently twice in PBS. Hereafter the binding of the construct was detected in two differnet ways: The construct as a hole was detected via its C-terminal Histidin Tag with a murine FITC conjugated anti-His-Tag antibody (Dianova, Hamburg, FRG, DIA920), diluted 1:20 in 50 μl PBS with 2% FCS. The correct expression of the 4.1 BB ligand domain was checked by using an R-Phycoerythrin-conjugated mouse anti human 4-1BB ligand antibody (BD biosciences, Heidelberg, FRG), diluted 1:10 in 50,μl PBS with 2% FCS (thick line). As negative control untransfected CHO cells were used (thin line). Cells were analyzed by flowcytometry on a FACS-scan (Becton Dickinson, Heidelberg). FACS staining and measuring of the fluorescence intensity were performed as described in Current Protocols in Immunology (Coligan, Kruisbeek, Margulies, Shevach and Strober, Wiley-lnterscience, 2002).

The binding ability of the anti-NKG2D binding domain and the anti-EpCAM binding domain respectively were clearly detectable as shown in FIG. 16.

Purification and Analysis of 4-1BB Ligand Trimer as Examplified by the Bispecific scFv—4-1BB Ligand Construct: scFv anti-NKG2D—scFv anti-EpCAM—Human 4-1BB Ligand Fusion Protein

The fusion proteins were isolated from cell culture supernatant in a two step purification process including immobilized metal affinity chromatography (IMAC), and gelfiltration. The final product had an apparent molecular weight of approx. 47 kDa (single chain fusion protein) or 70 kDa (bispecific single chain fusion protein) on SDS PAGE and Western Blot. The detected molecular weight of the fusion proteins, however, was approx. 150 kDa (single chain fusion protein) or 220 kDa (bispecific single chain fusion protein) under native conditions as determined by gelfiltration in PBS. That corresponds to a trimeric form of the fusion constructs. The purity of the isolated protein was in most cases>95% as determined by SDS-PAGE. The final yield of purified protein was approx. 400 μg/I cell culture supernatant. Äkta FPLC System and GradiFrac (Pharmacia, Tennenlohe, Germany) and Unicorn Software were used for chromatography. All chemicals were of research grade and purchased from Sigma (Deisenhofen, Germany) or Merck (Darmstadt, Germany).

IMAC was performed, using a HisTrap 5 ml column (Amersham Biosciences Europe GmbH, Freiburg, Germany) that was preloaded with NiSO₄ according to the manufacturers protocol. The column was equilibrated with buffer A2 (20 mM NaPP pH 7.2, 0.4 M NaCl). The sample was applied to the column with a flow rate of 1 ml/min and the column was washed with buffer A2 to remove unbound sample. Bound protein was eluted using a two step gradient of buffer B2 (20 mM NaPP pH 7.0, 0.4 M NaCl, 0.5 M Imidazol) Step 1: 10% buffer B2, step 2: 100% buffer B2, each step for 5 column volumes. Eluted protein fractions from the second step were pooled for further purification.

Gelfiltration chromatography was performed on a Sephadex S200 HiPrep column (Amersham Biosciences Europe GmbH, Freiburg, Germany) equilibrated with PBS (Gibco Invitrogen Corp., Carlsbad, USA). Eluted protein samples (flow rate 1 ml/min) were subjected to SDS-Page and Western Blot for detection of the fusion protein. The column was previously calibrated for molecular weight determination (molecular weight marker MW GF-200, Sigma-Aldrich Chemie GmbH, Munich, Germany). Protein concentrations were determined measuring the values at 280 nm in combination with the molar absorption coefficient or by using the Pierce microBCA kit (Pierce Biotechnology Inc., Rockford, IL, USA).

SDS PAGE under reducing conditions was performed with precast 4-12% Bis Tris gels (Invitrogen Corp., Carlsbad, USA). Sample preparation and application were according to the manufacturers protocol. The molecular weight was determined with MultiMark protein standard (Invitrogen Corp., Carlsbad, USA). The gel was stained with colloidal Coomassie (Invitrogen protocol).

Western Blot was performed with a BioTrace membrane (Pall Life Sciences, Dreieich, Germany) and the Invitrogen Blot Module according to the manufacturers protocol. The antibodies used were Penta His (Qiagen, Hilden, Germany) and Goat-anti-Mouse-AP (Sigma-Aldrich Chemie GmbH, Munich, Germany). The staining solution was BCIP/NBT liquid (Sigma-Aldrich Chemie GmbH, Munich, Germany).

The purification of scFv anti-NKG2D—scFv anti-EpCAM—human CD30 ligand fusion protein is shown in FIGS. 25, 26, and 27. From the gelfiltration and the Western blot data it becomes clear that the construct is present in trimeric form. The Western blot data indicate that half of the purified protein appears as a trimer and the other half is aggregated. But no monomer is detectable. The purity of the trimer is around 50%.

Assay for EpCAM Binding

Binding of the purified trifunctional construct to the extracellular portion of the EpCAM antigen was tested using a FACS assay. For that purpose the EpCAM positive human gastric cancer cell line Kato III (obtained from American Type Culture Collection (ATCC) Manassas, Va. 20108 USA, ATCC number: HTB-103) was used. Cells were cultured according to the recommendations of the supplier and a number of 2.5*10⁵ cells was incubated with 10 μg/ml of the construct in 50 μl PBS with 2% FCS. The binding of the construct was detected with an anti-His antibody (Penta-His Antibody, BSA free, obtained from Qiagen GmbH, Hilden, FRG) at 2 μg/ml in 50 μl PBS with 2% FCS. As a second step reagent a R-Phycoerythrin-conjugated affinity purified F(ab′)₂ fragment, goat anti-mouse IgG, Fc-gamma fragment specific antibody, diluted 1:100 in 50 μl PBS with 2% FCS (obtained from Dianova, Hamburg, Germany) was used. The samples were measured on a FACS scan (BD Biosciences, Heidelberg, Germany). EpCAM binding was clearly detectable (FIG. 28).

Binding Study of NKG2D on Freshly Isolated NK Cells

Mononuclear cells (PBMC) were prepared by Ficoll density centrifugation from 250 ml peripheral blood obtained from a healthy donor. NK cells with the typical phenotype CD16+CD56+ were purified from the peripheral blood of healthy donors with the NK Cell Isolation Kit II (MACS, Bergisch Gladbach, Germany) leading to negatively sorted, untouched fresh NK cells. The isolation procedure was performed according to the instructions of the manufacturer. Successful separation of NK cells was controlled by flow cytometry after single staining with an anti-CD1 6 antibody (FIG. 29F). The purity of the CD16+ NK cells proved to be 74%. Flow cytometric monitoring of the NKG2D staining was equally carried out by single stainings with a commercially available anti-NKG2D antibody (1D11) (BD Biosciences Pharmingen, Heidelberg, Germany), the anti-NKG2D mab clone 11B2D10 (Micromet AG, Munich, Germany as described in WO 0171005), which is the source of the anti-NKG2D single chain antibody portion within the described construct, and the cell culture supernatant of scFv anti-NKG2D (11B2D10)—scFv anti-EpCAM (4-7)—human 4-1BBL. The FACS binding analysis was performed as described previously.

Most of the isolated NK cells were bound by the anti-NKG2D antibody (1D11l ) (96%, FIG. 29C), the anti-NKG2D mab clone 11B2D10 (82%, FIG. 29D), and the cell culture supernatant of scFv anti-NKG2D (11B2D10)—scFv anti-EpCAM (4-7)—human 4-1BBL trispecific single chain construct (86%, FIG. 29E) demonstrating that the anti-NKG2D portion of the scFv anti-N KG2D (11B2D10)—scFv anti-EpCAM (4-7)—human 4-1BBL trispecific construct specifically binds NKG2D on NK cells. The controls for the FACS analyses depicted by the unstained NK cells (FIG. 29A) and the NK cells stained only by the secondary antibody (FIG. 29B) showed almost no or 10% staining.

### EXAMPLE 4

Generation of a scFv anti-EpCAM—Human 4-1BB Ligand

In order to generate a further construct consisting of an anti-EpCAM scFv designated M79 in combination with the human 4-1BB ligand the following cloning steps were performed. The anti-EpCAM scFv M79 as described in Mack, M. et al. (1995) Proc Natl Acad Sci USA 92, 7021-7025 was used as tumor targeting part and the construct described in this publication served as basis for generating the anti-EpCAM—4-1BBL construct. A variant without Flag sequence of this construct (Kufer et al., 1997, Cancer Immunol lmmunother 45, 193-197) was enzymatically digested with the restriction enzymes BspEl and Sall. The resulting vector (now without CD3 part) was fused with an appropriate digested DNA-fragment comprising the human 4.1 BB ligand as described in example 3. The resulting construct (SEQID 19 and 20) with the domain arrangement VL_{anti-EpCAM (M79)}—VH_{anti-EpCAM (M79)}—Extracellular Domain_{4.1BB ligand} was transfected and expressed in CHO cells according to the example 2. Purification was performed as described in Kufer et al., 2001, Cancer immunity Vol. 1, p. 10. The sequence and a schematic representation of this construct are shown in FIG. 17. SEQ ID NO: 19 and 20 show the sequence of the construct without His-tag.

Flow Cytometric Binding Analysis of the scFv anti-EpCAM—Human 4-1BB Ligand Construct

In order to test the functionality of the construct with regard to binding capability a FACS analysis was performed. For this purpose CHO transfectants expressing the extracellular domain of the EpCAM antigen were used. 200,000 EpCAM+ CHO cells were incubated with 50 μl the pure cell culture supernatants of CHO cells transfected with the anti-EpCAM—4-1BBL construct for 30 min on ice. The cells were washed subsequently twice in PBS. Finally, bound construct was detected in two different ways: The construct as a whole was detected via its C-terminal Histidin Tag with a murine FITC conjugated anti-His-Tag antibody (Dianova, Hamburg, FRG, DIA920), diluted 1:20 in 50 μl PBS with 2% FCS. The correct expression of the 4-1BBL domain was checked by using an R-Phycoerythrin-conjugated mouse anti human 4-1BB ligand antibody (BD biosciences, Heidelberg, FRG), diluted 1:10 in 50 μl PBS with 2% FCS (thick line). As negative control the cell culture supernatant containing the secreted anti-EpCAM—4-1BBL construct was not applied (thin line).

Cells were analyzed by flowcytometry on a FACS-scan (Becton Dickinson, Heidelberg). FACS staining and measuring of the fluorescence intensity were performed as described in Current Protocols in Immunology (Coligan, Kruisbeek, Margulies, Shevach and Strober, Wiley-lnterscience, 2002).

The binding ability of the anti-EpCAM binding domain and the presence of the 4-1BBL respectively were clearly detectable as shown in FIG. 18.

Purification and Analysis of 4-1BB Ligand Trimer as Examplified by the scFv anti-EpCAM (M79)—human 4-1BB Ligand Fusion Protein

The scFv anti-EpCAM (M79)—human 4-1BB ligand protein was isolated from cell culture supernatant in a three step purification process including cation exchange chromatography (FIG. 20), immobilized metal affinity chromatography (IMAC) (FIG. 21) and gelfiltration (FIG. 22). The final product had an apparent molecular weight of approx. 50 kDa on SDS PAGE (FIG. 23) and Western Blot (FIG. 24). In contrast to its 50 kDa size on SDS PAGE, the protein had a molecular weight of approx. 150 kDa under native conditions as determined by gelfiltration in PBS. This 150 kDa size corresponds to a trimeric form of scFv anti-EpCAM (M79)—human 4-1BB ligand. The purity of the isolated protein was>95% as determined by SDS-PAGE (FIG. 23). The final yield of purified protein was approx. 5.5 mg/l cell culture supernatant. Akta FPLC System and GradiFrac (Pharmacia, Tennenlohe, Germany) and Unicorn Software were used for chromatography. All chemicals were of research grade and purchased from Sigma (Deisenhofen, Germany) or Merck (Darmstadt, Germany).

In a first purification step cation exchange chromatography was performed on a SP Sepharose column (Pharmacia, Tennenlohe, Germany) that was equilibrated with buffer A1 (20 mM MES pH 5.5). Cell culture supernatant was diluted 1:3 with buffer A1 and applied to the column (bedsize 300 ml, packed in a XK column, Pharmacia, Tennenlohe, Germany, according to the manufacturers protocol) with a flow rate of 20 ml/min at 4° C. Unbound sample was washed out with buffer A1 and the bound protein was eluted with a three step gradient of 30%, 50% and 100% buffer B1 (20 mM MES pH 5.5, 1 M NaCl) in volumes of two column volumes (CV). Eluted protein fractions from the 50% B1 step were pooled for further purification (FIG. 20).

In a second purification step IMAC was performed, using a HisTrap 5 ml column (Pharmacia, Tennenlohe, Germany) that was preloaded with NiSO₄ according to the manufacturers protocol. The column was equilibrated with buffer A2 (20 mM NaPP pH 7.2, 0.4 M NaCl). The sample was applied to the column with a flow rate of 1 ml/min and the column was washed with buffer A2 to remove unbound sample. Bound protein was eluted using a three step gradient of buffer B2 (20 mM NaPP pH 7.0, 0.4 M NaCl, 0.5 M Imidazol). Step 1: 10% buffer B2, step 2: 30% buffer B2, step 3: 100% buffer B2, each step did encompass four column volumes. Eluted protein fractions from the second step were pooled for further purification.

In a third purification step gelfiltration chromatography was performed on a Sephadex S200 HiPrep column (Pharmacia, Tennenlohe, Germany) equilibrated with PBS (Gibco Invitrogen Corp., Carlsbad, USA). Eluted protein samples (flow rate 1ml/min) were subjected to SDS-Page and Western Blot for detection of bispecific scFv antibody (scFv anti-EpCAM—scFv anti-CD3). The column was previously calibrated for molecular weight determination (molecular weight marker kit MW GF-200, Sigma-Aldrich Chemie GmbH, Munich, Germany). Protein concentrations were determined using absorption values at 280 nm in combination with the molar absorption coefficient.

SDS PAGE was performed under reducing conditions with precast 4-12% Bis Tris gels (Invitrogen GmbH, Karlsruhe, Germany). Sample preparation and application were according to the manufacturers protocol. The molecular weight was determined with MultiMark protein standard (Invitrogen GmbH, Karlsruhe, Germany). The gel was stained with colloidal Coomassie according to Invitrogen protocol.

Western Blot was performed with a BioTrace membrane (Pall Gelman GmbH, Dreieich, Germany) and the Invitrogen Blot Module according to the manufacturers protocol. The antibodies used were Penta His (Qiagen, Hilden, Germany) and goat-anti-mouse-AP (Sigma-Aldrich Chemie GmbH, Munich, Germany), the stainer was BCIP/NBT liquid (Sigma-Aldrich Chemie GmbH, Munich, Germany).

### EXAMPLE 5

Priming Assay with the B7.1—scFv anti-EpCAM—Human 4-1BB Ligand Construct

Direct priming of naive human CD4+ T cells was investigated using the 4-1BBL—anti-EpCAM scFv—B7.1 construct.

The construct described in example 2A was used to specifically target the costimulatory molecule 4-1BBL to the epithelial cell adhesion molecule (EpCAM), a surface antigen successfully used as target for antibody therapy of minimal residual colorectal cancer. T cell priming was monitored by flow cytometric analysis of CD45 isoform expression.

Purification of Naive T Cells

Naive CD4+ and CD8+ T lymphocytes with the typical phenotype CD45RA+ RO— were purified from the peripheral blood of healthy donors. Mononuclear cells (PBMC) were prepared by Ficoll density centrifugation from 500 ml peripheral blood obtained from a healthy donor. CD4+ and CD8+ T cells were isolated by negative selection using commercially available cell separation kits (R&D Systems, HCD4C-1000 and HCDBC-1000 respectively, Wiesbaden, Germany). Each CD4+ or CD8+ T cell column was loaded with 2×108 PBMC which had been preincubated with the manufacturer's antibody cocktail, except that the CD8+ T cell cocktail was supplemented with 1 μg monoclonal anti-CD11b antibody (Coulter, Krefeld, Germany) per column. Successful separation of CD4+ and CD8+ T cells was controlled by flow cytometry after single staining with an anti-CD4 or anti-CD8 antibody respectively. Absence of CD11b+ cells from CD8+ T cell preparations was confirmed by single staining with an anti-CD28 antibody, since CD11b+ CD8+ T cells are known to be CD28— and vice versa.

CD45RO+ cells were removed from purified CD4+ or CD8+ T cells by incubation with a murine monoclonal anti-CD45RO antibody UCHL-1, 31301 (PharMingen, Heidelberg, Germany), followed by separation using magnetic beads conjugated with a polygonal sheep anti-mouse lg antibody (Dynal, Hamburg, Germany). For depletion of residual antigen presenting cells (e.g. dendritic cells) purified CD4+ or CDB+ T cells were coincubated with murine monoclonal antibodies against CD45RO and HLA-DR, DP, DQ (PharMingen, Heidelberg, Germany) prior to incubation with magnetic anti-mouse Ig beads. The purity of the remaining naive CD4+ or CD8+ T cells proved to be 95 to 97% as determined by flow cytometry after double staining with anti-CD45RA and anti-CD45RO. The yields of naive T cells were 2 to 3×10⁷ (CD4) and 5×10⁶ (CD8) per 500 ml peripheral blood.

Flow Cytometry

Flow cytometric analysis of CD45 isoform expression was carried out by double staining of 1×10⁵ cells with a PE-conjugated monoclonal anti-CD45RA antibody (Coulter, Krefeld, Germany) and a FITC-conjugated monoclonal anti-CD45RO antibody UCHL-1, F 0800 (DAKO, Hamburg, Germany) for 30 minutes on ice. Flow cytometric monitoring of T cell purification was equally carried out by single stainings with a Tricolor-conjugated monoclonal anti-CD4 antibody (MHCD0406), a Tricolor-conjugated monoclonal anti-CD8 antibody (MHC0806) and a FITC-conjugated monoclonal anti-CD28 antibody (MHCD2801), all from Medac (Hamburg, Germany).

Priming Assay

Naive CD4+ T lymphocytes with the typical phenotype CD45RA+RO— were purified from the peripheral blood of healthy donors and incubated with irradiated EpCAM-transfected CHO cells as stimulator cells (according to Kufer et al., 2001, Cancer Immunity 1, 10).

The primary signal was mediated by the bispecific single-chain antibody (bscAb) EpCAM (M79)×CD3 (Kufer et al., 1997, Cancer Immunol Immunother 45, 193-197) imitating specific antigen recognition through the TCR; the second or costimulatory signal was mediated by an EpCAM specific B7.1 construct (B7.1—scFv antiEpCAM, Kufer et al., 2001, Cancer Immunity 1, 10). T cell priming was monitored by flow cytometry on day 6 by simultaneously measuring the expression of CD45RA and CD45RO.

In the presence of the bispecific single-chain antibody (bscAb) EpCAM (M79)×CD3 alone at a concentration of 250 ng/ml (FIG. 19A) and at a concentration of 50 ng/ml (FIG. 19D) cells stayed unprimed displaying the phenotype CD45RA+RO—. In the presence of both EpCAM-specific B7.1 construct (500 ng/ml) and bscAb EpCAM×CD3 (250 ng/ml), the CD45 phenotype of almost the entire population of naive T cells changed to that of primed T cells, i.e. CD45RA-RO+, within 6 days (FIG. 15B). At a suboptimal concentration of bispecific single-chain antibody (bscAb) EpCAM (M79)×CD3 (50 ng/ml) and a concentration of 500 ng/ml EpCAM specific B7.1 construct (B7.1—scFv antiEpCAM, Kufer et al., 2001, Cancer Immunity 1, 10) the CD45 phenotype of a minor part of the population of naive T cells (5, 7%) changed to that of primed T cells, i.e. CD45RA-RO+, within 6 days (FIG. 19E).

However, adding the B7.1—scFv anti-EpCAM—hu4-1BB ligand construct of example 2 at a concentration of 500 ng/ml together with the bispecific single-chain antibody (bscAb) EpCAM (M79)×CD3 at the suboptimal concentration of 50 ng/ml changed the CD45 phenotype of a substantially increased part of the population of naive T cells (24%) to that of primed T cells, i.e. CD45RA-RO+, within 6 days (FIG. 19F). This result demonstrates that the priming with the B7.1—scFv anti-EpCAM—4-1BB ligand construct works better than with a B7.1—scFv anti-EpCAM construct (Kufer et al., 2001, Cancer Immunity 1, 10).

Importantly, the combination of scFv anti-EpCAM—4-1BB ligand (see example 4) construct and bscAb EpCAM×CD3 could not induce substantial changes in CD45 isoform expression in the absence of B7.1 costimulation (FIG. 19C).

All constructs were tested alone on themselves for T cell priming, but none of them showed changed CD45 phenotype from naive T cells to that of primed T cells, i.e. CD45RA-RO+, within 6 days (FIGS. 19G, H, I): Neither the B7.1—scFv anti-EpCAM construct (FIG. 19G), nor the scFv anti-EpCAM—4-1BB ligand construct (FIG. 19H), nor the B7.1—scFv anti-EpCAM -hu4-1BB ligand construct (FIG. 19I).

Cell culture was carried out at 37° C. and 6% CO2 for all priming experiments.

1. A trimeric polypeptide construct, wherein each monomer of the trimeric polypeptide construct consists of two or three domains, and wherein the first domain is the extracellular domain of 4-1BBL or (a) part(s) thereof, the second domain comprises an antigen-interaction-site which is located N terminally of the first domain and, optionally, the third domain combines said first and second domain via a peptide linker, wherein said peptide linker does not comprise any polymerization activity.

2. The trimeric polypeptide construct of claim 1, wherein said extracellular domain is the complete extracellular domain of 4-1BBL.

3. The trimeric polypeptide construct of claim 1, wherein said antigen interaction-site comprises at least two domains which specifically interact with separate antigens.

4. The trimeric polypeptide construct of claim 3, wherein said at least two domains are combined via a peptide linker

5. The trimeric polypeptide construct of any of claim 1, wherein said antigen-interaction-site is specific for one or more cell surface marker.

6. The trimeric polypeptide construct of claim 5, wherein said cell surface marker is a tumor marker.

7. The trimeric polypeptide construct of any one of claim 1, wherein said antigen-interaction-site comprises at least one domain which is an antibody derived region.

8. The trimeric polypeptide construct of any one of claim 1, wherein said antigen-interaction-site comprises at least two antibody-derived regions.

9. The trimeric polypeptide construct of any one of claim 1, wherein said antigen-interaction-site comprises the extracellular domain of a member of the B7 family or a fragment or a derivative thereof which is capable of binding to its specific receptor.

10. The trimeric polypeptide construct of any one of claim 7, wherein said antigen-interaction-site is selected from the group consisting of scFv, Fab, and single lo variable regions.

11. The trimeric polypeptide construct of claim 10, wherein said scFv is selected from the group consisting of scFv specific for EpCAM, NKG2D, CD19, PSMA, MCSP, stn (TAG72), CD44v6, carbonic anhydrase IX (CAIX) , CEA, EGFR, CD33, Wue-1, CD3, Muc-1, CD20, Her2-neu, Her 3, Her 4 and Lewis-Y.

12. The trimeric polypeptide construct of any one of claim 9, wherein said member of the B7 family or a fragment or a derivative thereof is selected from the group consisting of B7.1, B7.2, B7-H3, B7- RP1, B7-DC, PDL1and PDL2.

13. The trimeric polypeptide construct of any one of claim 10, wherein said second domain of each monomer comprises a scFv specific for EpCAM.

14. The trimeric polypeptide construct of claim 13, wherein each monomer has the amino acid sequence as shown in SEQ ID NO: 20.

15. The trimeric polypeptide construct of any one of claim 10, wherein said second domain of each monomer comprises a scFv from/or derived from the monoclonal antibody 237.

16. The trimeric polypeptide construct of claim 15, wherein each monomer has the amino acid sequence as shown in SEQ ID NO: 8.

17. The trimeric polypeptide construct of any one of claim 1, wherein said second domain of each monomer comprises a scFv specific for EpCAM and a scFv specific for NKG2D.

18. The trimeric polypeptide construct of claim 17, wherein each monomer has the amino acid sequence as shown in SEQ ID NO: 18.

19. The trimeric polypeptide construct of any one of claim 11, wherein said second domain of each monomer comprises a bispecific scFv construct wherein at least on scFv is specific for CD3.

20. The trimeric polypeptide construct of claim 19, wherein the scFv in each monomer which is specific for CD3 has the amino acid sequence as shown in SEQ ID NO: 22.

21. The trimeric polypeptide construct of any of 12, wherein said second domain of each monomer comprises a scFv specific for EpCAM and an antigen interaction site which is the extracellular domain of B7.1 or a fragment or a derivative thereof which is capable of binding to its specific receptor.

22. The trimeric polypeptide construct: of claim 21, wherein each monomer has the amino acid sequence as shown in SEQ ID NO: 16.

23. The trimeric polypeptide construct of any one of claim 1, consisting of at least two different monomers, wherein said different monomers are characterized by of different antigen-interaction-sites.

24. The trimeric polypeptide construct of any one of claim 1, wherein at least one monomer further comprises a tag.

25. The trimeric polypeptide construct of claim 24, wherein said tag is a HIS-tag at the C-terminus of the at least one monomer.

26. The trimeric polypeptide construct of any one of claim 1, wherein said polypeptide construct is expressed in a eukaryotic expression system.

27. A nucleic acid molecule encoding a monomer of a trimeric polypeptide construct of claim 1.

28. A vector comprising the nucleic acid molecule of claim 27.

29. The vector of claim 28, wherein the nucleic acid molecule is DNA.

30. The vector of claim 28 which is an expression vector wherein the nucleic acid molecule encoding a monomer of a trimeric polypeptide construct of claim 1 is operatively linked to one or more control sequences allowing the transcription and optionally expression in prokaryotic and/or eukaryotic hosts.

31. The vector of claim 30, which is pEF-DHFR or pEF-ADA.

32. A host containing at least one vector of claim 28 or at least one nucleic acid molecule of claim 27.

33. The host of claim 32 which is a bacteria, an insect, fungal, plant or animal cell.

34. The host of claim 32 which is a mammalian cell.

35. The host of claim 34 which is a human cell or human cell line.

36. A process for the production of a trimeric polypeptide construct said process comprising culturing a host of claim 32 under conditions allowing the expression of the polypeptide construct and recovering the produced polypeptide construct from the culture.

37. The process of claim 36, wherein said expression leads to an rate of trimerization of at least 90% and recovering the produced polypeptide construct from the culture.

38. A composition comprising a trimeric polypeptide construct of claim 1 or as produced by the process of claim 36, a nucleic acid molecule of claim 27, a vector of claim 28 or a host of claim 32 and, optionally, a proteinaceous compound capable of providing an activation signal for immune effector cells.

39. The composition of claim 38 which is a pharmaceutical composition further comprising, optionally suitable formulations of carrier, stabilizers and/or excipients.

40. The composition of claim 39 which is a diagnostic composition further comprising, optionally, means and methods for detection.

41. Use of a trimeric polypeptide construct of claim 1 or as produced lay the process of claim 36, a nucleic acid molecule of claim 27, a vector of claim 28 or a host of claim 32 for the preparation of a pharmaceutical composition for the prevention, treatment or amelioration of a proliferative disease, a tumorous disease, an inflammatory disease, an immunological disorder, an autoimmune disease, an infectious disease, viral disease, allergic reactions, parasitic reactions, graft-versus-host diseases or host-versus- graft diseases.

42. The use of claim 41, wherein said tumorous disease is epithelial cancer or a minimal residual cancer.

43. A method for the prevention, treatment or amelioration of a proliferative disease, a tumorous disease, an inflammatory disease, an immunological disorder, an autoimmune disease, an infectious disease, viral disease, allergic reactions, parasitic reactions, graft-versus-host diseases or host- versus-graft diseases comprising the step of administering to a subject in need of such a prevention, treatment or amelioration a trimeric polypeptide construct of claim 1 or as produced by the process of claim 36, a nucleic acid molecule of claim 27, a vector of claim 28 or a host of claim 32.

44. The method of claim 43, wherein said tumorous disease is epithelial cancer or a minimal residual cancer.

45. The method of claim 43, wherein said subject is a human.

46. The method of claim 43 further comprising, the administration of a proteinaceous compound capable of providing an activation signal for immune effecter cells

47. The method of claim 46, wherein said proteinaceous compound is administered simultaneously or non- simultaneously with a trimeric polypeptide construct of claim 1 or as produced by the process of claim 36, a nucleic acid molecule of claim 27, a vector of claim 28 or a host of claim 32.

48. A kit comprising a trimeric polypeptide construct of claim 1 or as produced by the process of claim 36, a nucleic acid molecule of claim 27, a vector of claim 28 or a host of claim 32.


SEQUENCE LISTING (22 sequences)

SEQ ID NO: 1 - DNA, 23 bp, Artificial sequence
  5′ murine murine 4-1BBL primer
cgggatcccgcaccgagcctcgg

SEQ ID NO: 2 - DNA, 31 bp, Artificial sequence
  3′ murine 4-1BBL primer
ggatccggattcccatgggttgtcgggtttc

SEQ ID NO: 3 - DNA, 625 bp, mus musculus
cccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatccgg

SEQ ID NO: 4 - protein, 208 aa, mus musculus
RTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWESG

SEQ ID NO: 5 - DNA, 732 bp, Artificial sequence
  scFv anti237 nucleic acid sequence
gatatccagctgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagcgtggaggctgaggatctgggagtttatttctgctctcaaagtacacatgttcccacgttcggaggggggaccaagctcgagatcaaaggtggtggtggttctggcggcggcggctccggtggtggtggttctcaggtgcaactgcagcagtctggaggtggcttggtgcaacctggaggatccatgaaaatcttttgtgctgcttctggattcacttttagtgatgcctggatggactgggtccgccagtctccagagaaggggcttgagtgggttgctgaaattagaaacaaagctaataatcatgaaacatattatgctgagtctgtgaaagggaggttcaccatcacaagagatgattccaaaagtagaatgtccctgcaaatgaacagcttaagagctgaagacactggcatttattactgttcgggggggaaggtacggaatgcttactggggccaagggaccacggtcaccgtctcctcc

SEQ ID NO: 6 - protein, 244 aa, Artificial sequence
  scFv anti237 amino acid sequence
DIQLTQSPLSLPVSLGDQASISCRSSQSLVHSNGNTYLHWYLQKPGQSPKLLIYKVSNRFSGVPDRFSGSGSGTDFTLKISSVEAEDLGVYFCSQSTHVPTFGGGTKLEIKGGGGSGGGGSGGGGSQVQLQQSGGGLVQPGGSMKIFCAASGFTFSDAWMDWVRQSPEKGLEWVAEIRNKANNHETYYAESVKGRFTITRDDSKSRMSLQMNSLRAEDTGIYYCSGGKVRNAYWGQGTTVTVSS

SEQ ID NO: 7 - DNA, 1449 bp, Artificial sequence
  scFv anti237 - 4-1BBL nucleic acid sequence
atgggatggagctgtatcatcctcttcttggtagcaacagctacaggtgtacactccgatatccagctgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagcgtggaggctgaggatctgggagtttatttctgctctcaaagtacacatgttcccacgttcggaggggggaccaagctcgagatcaaaggtggtggtggttctggcggcggcggctccggtggtggtggttctcaggtgcaactgcagcagtctggaggtggcttggtgcaacctggaggatccatgaaaatcttttgtgctgcttctggattcacttttagtgatgcctggatggactgggtccgccagtctccagagaaggggcttgagtgggttgctgaaattagaaacaaagctaataatcatgaaacatattatgctgagtctgtgaaagggaggttcaccatcacaagagatgattccaaaagtagaatgtccctgcaaatgaacagcttaagagctgaagacactggcatttattactgttcgggggggaaggtacggaatgcttactggggccaagggaccacggtcaccgtctcctccggaggtggtggatcccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatccgggcatcatcaccatcatcattag

SEQ ID NO: 8 - protein, 457 aa, Artificial sequence
  scFv anti237 - 4-1BBL amino acid sequence
DIQLTQSPLSLPVSLGDQASISCRSSQSLVHSNGNTYLHWYLQKPGQSPKLLIYKVSNRFSGVPDRFSGSGSGTDFTLKISSVEAEDLGVYFCSQSTHVPTFGGGTKLEIKGGGGSGGGGSGGGGSQVQLQQSGGGLVQPGGSMKIFCAASGFTFSDAWMDWVRQSPEKGLEWVAEIRNKANNHETYYAESVKGRFTITRDDSKSRMSLQMNSLRAEDTGIYYCSGGKVRNAYWGQGTTVTVSSGGGGSRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWESG

SEQ ID NO: 9 - DNA, 26 bp, Artificial sequence
  5′ human 4-1BBL primer
cgggatccctcgcctgcccctgggcc

SEQ ID NO: 10 - DNA, 30 bp, Artificial sequence
  3′ human 4-1BBL primer
ggatccggattccgacctcggtgaagggag

SEQ ID NO: 11 - DNA, 629 bp, Homo sapiens
ggatccctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaatccgg

SEQ ID NO: 12 - protein, 208 aa, Homo sapiens
LACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESG

SEQ ID NO: 13 - DNA, 26 bp, Artificial sequence
  5′ scFv anti-EpCAM (4-7) primer
cattttcctgataactccggaggtgg

SEQ ID NO: 14 - DNA, 30 bp, Artificial sequence
  3′ scFv anti-EpCAM (4-7) primer
aagtccggatttgatctcaagcttggtccc

SEQ ID NO: 15 - DNA, 2160 bp, Artificial sequence
  B7.1-scFv antiEpCAM-hu4-1BBL nucleic acid sequence
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataactccggaggtggtggatccgaggtgcagctgctcgagcagtctggagctgagctggcgaggcctggggcttcagtgaagctgtcctgcaaggcttctggctacaccttcacaaactatggtttaagctgggtgaagcagaggcctggacaggtccttgagtggattggagaggtttatcctagaattggtaatgcttactacaatgagaagttcaagggcaaggccacactgactgcagacaaatcctccagcacagcgtccatggagctccgcagcctgacctctgaggactctgcggtctatttctgtgcaagacggggatcctacgatactaactacgactggtacttcgatgtctggggccaagggaccacggtcaccgtctcctcaggtggtggtggttctggcggcggcggctccggtggtggtggttctgagctcgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagcttgagatcaaatccggaggtggtggatccctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaatccgggcatcatcaccatcatcattga

SEQ ID NO: 16 - protein, 687 aa, Artificial sequence [sequence omitted]

SEQ ID NO: 17 - DNA, 2214 bp, Artificial sequence
  anti-NKG2D - anti-EpCAM - human 4-1 BBL nucleic acid sequence
gaattcaccatgggatggagctgtatcatcctcttcttggtagcaacagctacaggtgtacactccgatatccagctgacccagtctccagcctccctatctgcatctgtgggagaaactgtcaccatcacatgtcgagcaagtgggaatattcacaattatttagcttggtatcagcagaaacagggaaaatctcctcaggtcctggtctataatgcaaaaaccttagcagatggtgtgccatcaaggttcagtggcagtggatcaggaacacaatattccctcaagatcaacagcctgcagcctgaagattttgggagttattactgtcaacatttttggagtactacgtggacgttcggtggagggaccaagctcgagatcaaaggtggtggtggttctggcggcggcggctccggtggtggtggttctcaggtccaactgcagcagtctggggctgagctggtgaggcctggggcttcagtgaagctgtcctgcaaggcttctggctacacgttcaccagctactggatgaactgggttcagcagaggcctgagcaaggccttgagtggattggaaggattgatccttacgatagtgaaactcactacaatcaaaagttcaaggacaaggccatattgactgtagacaaatccgccagcacagcctacatgcaactcagcagcctgacatctgaggactctgcggtctattactgtgcaaaaatgggtgattactcctttgactactggggccaagggaccacggtcaccgtctcctccggaggtggtggatccgaggtgcagctgctcgagcagtctggagctgagctggcgaggcctggggcttcagtgaagctgtcctgcaaggcttctggctacaccttcacaaactatggtttaagctgggtgaagcagaggcctggacaggtccttgagtggattggagaggtttatcctagaattggtaatgcttactacaatgagaagttcaagggcaaggccacactgactgcagacaaatcctccagcacagcgtccatggagctccgcagcctgacctctgaggactctgcggtctatttctgtgcaagacggggatcctacgatactaactacgactggtacttcgatgtctggggccaagggaccacggtcaccgtctcctcaggtggtggtggttctggcggcggcggctccggtggtggtggttctgagctcgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagcttgagatcaaatccggaggtggtggatccctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaatccgggcatcatcaccatcatcattga

SEQ ID NO: 18 - protein, 709 aa, Artificial sequence
  anti-NKG2D - anti-EpCAM - human 4-1 BBL amino acid sequence
DIQLTQSPASLSASVGETVTITCRASGNIHNYLAWYQQKQGKSPQVLVYNAKTLADGVPSRFSGSGSGTQYSLKINSLQPEDFGSYYCQHFWSTTWTFGGGTKLEIKGGGGSGGGGSGGGGSQVQLQQSGAELVRPGASVKLSCKASGYTFTSYWMNWVQQRPEQGLEWIGRIDPYDSETHYNQKFKDKAILTVDKSASTAYMQLSSLTSEDSAVYYCAKMGDYSFDYWGQGTTVTVSSGGGGSEVQLLEQSGAELARPGASVKLSCKASGYTFTNYGLSWVKQRPGQVLEWIGEVYPRIGNAYYNEKFKGKATLTADKSSSTASMELRSLTSEDSAVYFCARRGSYDTNYDWYFDVWGQGTTVTVSSGGGGSGGGGSGGGGSELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKSGGGGSLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESG

SEQ ID NO: 19 - DNA, 1380 bp, Artificial sequence
  scFv antiEpCAM-hu4-1BBL nucleic acid sequence
gatatccagctgacccagtctccaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgctcacgttcggtgctgggaccaagctcgagatcaaaggtggtggtggttctggcggcggcggctccggtggtggtggttctgatatcaagctgcaggagtctggacctggcctagtgcagccctcacagagcctgtccatcacctgcacagtctctggtttctcattaactagctatggtgtacactgggttcgccagtctccaggaaagggtctggagtggctgggagtgatatggagtggtggaagcacagactataatgcagctttcatatccagactgagcatcagcaaggacaattccaagagccaagttttctttaaaatgaacagtctgcaagctaatgacacagccatatattactgtgccagaatggagaactggtcgtttgcttactggggccaagggaccacggtcaccgtctcctccggaggtggtggatccctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaatccgggcatcatcaccatcatcattgagtcgac

SEQ ID NO: 20 - protein, 451 aa, Artificial sequence
  scFv antiEpCAM-hu4-1BBL amino acid sequence
DIQLTQSPKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLEIKGGGGSGGGGSGGGGSDIKLQESGPGLVQPSQSLSITCTVSGFSLTSYGVHWVRQSPGKGLEWLGVIWSGGSTDYNAAFISRLSISKDNSKSQVFFKMNSLQANDTAIYYCARMENWSFAYWGQGTTVTVSSGGGGSLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESG

SEQ ID NO: 21 - DNA, 729 bp, Artificial sequence
  scFv antiCD3 nucleic acid sequence
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 22 - protein, 243 aa, Artificial sequence
  scFv antiCD3 amino acid sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK